SEQUENCE LISTING (20 sequences)

SEQ ID NO: 1 - DNA, 46 bp, Artificial
  Synthetic - made in laboratory
gatccggccctgagggccgcagatggcccataccttcaaatattag

SEQ ID NO: 2 - DNA, 53 bp, Artificial
  Synthetic
ccgccggagcctccgccactagtgcccgagctccccttctgacgtttcctctg

SEQ ID NO: 3 - DNA, 58 bp, Artificial
  Synthetic
gcactagtggcggaggctccggcggtggcattcttaaaattctgaacgaaatagcatc

SEQ ID NO: 4 - DNA, 44 bp, Artificial
  Synthetic
atgcggccgcataggccttatagaccagttacctcatgaaaatc

SEQ ID NO: 5 - DNA, 2646 bp, Artificial
  Synthetic
ctcgagaaatcataaaaaatttatttgctttgtgagcggataacaattataatagattcaattgtgagcggataacaatttcacacagaattcattaaagaggagaaattaactatgagaggatctcaccatcaccatcaccatacggatccggccctgagggccgcagatggcccataccttcaaatattagagcaacctaaacagagaggatttcgtttccgttatgtatgtgaaggcccatcccatggtggattacctggtgcctctagtgaaaagaacaagaagtcttaccctcaggtcaaaatctgcaactatgtgggaccagcaaaggttattgttcagttggtcacaaatggaaaaaatatccacctgcatgcccacagcctggtgggaaaacactgtgaggatgggatctgcactgtaactgctggacccaaggacatggtggtcggcttcgcaaacctgggtatacttcatgtgacaaagaaaaaagtatttgaaacactggaagcacgaatgacagaggcgtgtataaggggctataatcctggactcttggtgcaccctgaccttgcctatttgcaagcagaaggtggaggggaccggcagctgggagatcgggaaaaagagctaatccgccaagcagctctgcagcagaccaaggagatggacctcagcgtggtgcggctcatgtttacagcttttcttccggatagcactggcagcttcacaaggcgcctggaacccgtggtatcagacgccatctatgacagtaaagcccccaatgcatccaacttgaaaattgtaagaatggacaggacagctggatgtgtgactggaggggaggaaatttatcttctttgtgacaaagttcagaaagatgacatccagattcgattttatgaagaggaagaaaatggtggagtctgggaaggatttggagatttttcccccacagatgttcatagacaatttgccattgtcttcaaaactccaaagtataaagatattaatattacaaaaccagcctctgtgtttgtccagcttcggaggaaatctgacttggaaaccagtgaaccaaaacctttcctctactatcctgaaatcaaagataaagaagaagtgcagaggaaacgtcagaaggggagctcgggcactagtggcggaggctccggcggtggcattcttaaaattctgaacgaaatagcatctattggttcaactaaacagaagcaagcaattcttgaaaagaataaagataatgaattgcttaaacgagtatatcgtctgacttattctcgtgggttacagtattatatcaagaaatggcctaaacctggtattgctacccagagttttggaatgttgactcttaccgatatgcttgacttcattgaattcacattagctactcggaaattgactggaaatgcagcaattgaggaattaactggatatatcaccgatggtaaaaaagatgatgttgaagttttgcgtcgagtgatgatgcgagaccttgaatgtggtgcttcagtatctattgcaaacaaagtttggccaggtttaattcctgaacaacctcaaatgctcgcaagttcttatgatgaaaaaggcattaataagaatatcaaatttccagcctttgctcagttaaaagctgatggagctcggtgttttgctgaagttagaggtgatgaattagatgatgttcgtcttttatcacgagctggtaatgaatatctaggattagatcttcttaaggaagagttaattaaaatgaccgctgaagcccgccagattcatccagaaggtgtgttgattgatggcgaattggtataccatgagcaagttaaaaaggagccagaaggcctagattttctttttgatgcttatcctgaaaacagtaaagctaaagaattcgccgaagttgctgaatcacgtactgcttctaatggaatcgccaataaatctttaaagggaaccatttctgaaaaagaagcacaatgcatgaagtttcaggtctgggattatgtcccgttggtagaaatatacagtcttcctgcatttcgtttgaaatatgatgtacgtttttctaaactagaacaaatgacatctggatatgataaagtaattttaattgaaaaccaggtagtaaataacctagatgaagctaaggtaatttataaaaagtatattgaccaaggtcttgaaggtattattctcaaaaatatcgatggattatgggaaaatgctcgttcaaaaaatctttataaatttaaagaagtaattgatgttgatttaaaaattgtaggaatttatcctcaccgtaaagaccctactaaagcgggtggatttattcttgagtcagagtgtggaaaaattaaggtaaatgctggttcaggcttaaaagataaagccggtgtaaaatcgcatgaacttgaccgtactcgcattatggaaaaccaaaattattatattggaaaaattctagagtgcgaatgcaacggttggttaaaatctgatggccgcactgattacgttaaattatttcttccgattgcgattcgtttacgtgaagataaaactaaagctaatacattcgaagatgtatttggtgattttcatgaggtaactggtctataa

SEQ ID NO: 6 - protein, 843 aa, Artificial
  Synthetic Construct
MRGSHHHHHHTDPALRAADGPYLQILEQPKQRGFRFRYVCEGPSHGGLPGASSEKNKKSYPQVKICNYVGPAKVIVQLVTNGKNIHLHAHSLVGKHCEDGICTVTAGPKDMVVGFANLGILHVTKKKVFETLEARMTEACIRGYNPGLLVHPDLAYLQAEGGGDRQLGDREKELIRQAALQQTKEMDLSVVRLMFTAFLPDSTGSFTRRLEPVVSDAIYDSKAPNASNLKIVRMDRTAGCVTGGEEIYLLCDKVQKDDIQIRFYEEEENGGVWEGFGDFSPTDVHRQFAIVFKTPKYKDINITKPASVFVQLRRKSDLETSEPKPFLYYPEIKDKEEVQRKRQKGSSGTSGGGSGGGILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 7 - DNA, 2505 bp, Artificial
  Synthetic
ctcgagaaatcataaaaaatttatttgctttgtgagcggataacaattataatagattcaattgtgagcggataacaatttcacacagaattcattaaagaggagaaattaactatgagaggatctcaccatcaccatcaccatacggatccggccctgagggccgcccgtgccaaggctaaagatcagacggacggtatctacgcagcgttcgacaccctgatgagcacggcgggtgttgactctcagattgcggcactggctgcgagcgaagccgacgccggcaccctggatgcggcgttgacgcaatcgctgcaagaggcgcagggccgctggggtctgggcctgcaccacttgcgccatgaggcgcgtttgaccgatgatggtgacattgagatcctgaccgacggtcgccctagcgcacgcgttagcgagggttttggtgccctggcgcaagcctacgcaccgatgcaagcgctggatgaacgtggcctgtcccaatgggctgcgctgggcgagggttatcgcgcaccgggcgatctgccgctggctcagctgaaggttctgatcgaacatgcccgcgatttcgaaactgattggagcgcgggtcgtggcgaaacgttccaacgcgtctggcgtaaaggtgacaccctgtttgtcgaggtggcgcgtccggcatccgccgaagcggcgctgagcgacgcggcgtgggatgtgattgccagcatcaaggaccgtgcgttccagcgtgagctgatgcgtcgttctgagaaagacggcatgctgggtgcgctgttgggtgctcgccatgctggcgccaaggcaaacttggcacagctgccagaggcccactttaccgtgcaggcatttgttcagactctgagcggtgcggcagcacgtaatgcggaagaatatcgcgcagcgctgaaaacggcggcagccgctctggaagagtaccagggcgtgaccacccgccagctgagcgaagtcctgcgtcacggtctgcgtgagagcggcactagtggcggaggctccggcggtggcattcttaaaattctgaacgaaatagcatctattggttcaactaaacagaagcaagcaattcttgaaaagaataaagataatgaattgcttaaacgagtatatcgtctgacttattctcgtgggttacagtattatatcaagaaatggcctaaacctggtattgctacccagagttttggaatgttgactcttaccgatatgcttgacttcattgaattcacattagctactcggaaattgactggaaatgcagcaattgaggaattaactggatatatcaccgatggtaaaaaagatgatgttgaagttttgcgtcgagtgatgatgcgagaccttgaatgtggtgcttcagtatctattgcaaacaaagtttggccaggtttaattcctgaacaacctcaaatgctcgcaagttcttatgatgaaaaaggcattaataagaatatcaaatttccagcctttgctcagttaaaagctgatggagctcggtgttttgctgaagttagaggtgatgaattagatgatgttcgtcttttatcacgagctggtaatgaatatctaggattagatcttcttaaggaagagttaattaaaatgaccgctgaagcccgccagattcatccagaaggtgtgttgattgatggcgaattggtataccatgagcaagttaaaaaggagccagaaggcctagattttctttttgatgcttatcctgaaaacagtaaagctaaagaattcgccgaagttgctgaatcacgtactgcttctaatggaatcgccaataaatctttaaagggaaccatttctgaaaaagaagcacaatgcatgaagtttcaggtctgggattatgtcccgttggtagaaatatacagtcttcctgcatttcgtttgaaatatgatgtacgtttttctaaactagaacaaatgacatctggatatgataaagtaattttaattgaaaaccaggtagtaaataacctagatgaagctaaggtaatttataaaaagtatattgaccaaggtcttgaaggtattattctcaaaaatatcgatggattatgggaaaatgctcgttcaaaaaatctttataaatttaaagaagtaattgatgttgatttaaaaattgtaggaatttatcctcaccgtaaagaccctactaaagcgggtggatttattcttgagtcagagtgtggaaaaattaaggtaaatgctggttcaggcttaaaagataaagccggtgtaaaatcgcatgaacttgaccgtactcgcattatggaaaaccaaaattattatattggaaaaattctagagtgcgaatgcaacggttggttaaaatctgatggccgcactgattacgttaaattatttcttccgattgcgattcgtttacgtgaagataaaactaaagctaatacattcgaagatgtatttggtgattttcatgaggtaactggtctataa

SEQ ID NO: 8 - protein, 796 aa, Artificial
  Synthetic Construct
MRGSHHHHHHTDPALRAARAKAKDQTDGIYAAFDTLMSTAGVDSQIAALAASEADAGTLDAALTQSLQEAQGRWGLGLHHLRHEARLTDDGDIEILTDGRPSARVSEGFGALAQAYAPMQALDERGLSQWAALGEGYRAPGDLPLAQLKVLIEHARDFETDWSAGRGETFQRVWRKGDTLFVEVARPASAEAALSDAAWDVIASIKDRAFQRELMRRSEKDGMLGALLGARHAGAKANLAQLPEAHFTVQAFVQTLSGAAARNAEEYRAALKTAAAALEEYQGVTTRQLSEVLRHGLRESGTSGGGSGGGILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 9 - DNA, 1842 bp, Artificial
  Synthetic
ctcgagaaatcataaaaaatttatttgctttgtgagcggataacaattataatagattcaattgtgagcggataacaatttcacacagaattcattaaagaggagaaattaactatgagaggatctcaccatcaccatcaccatacggatccggccctgagggcgactgttaaattcaaatacaaaggtgaggagaaagaagttgacatttccaagatcaaaaaggtatggcgcgtgggcaaaatgatctctttcacttacgacgaaggcggcggtaaaaccggtcgtggcgcggtgagcgaaaaggacgcgccaaaagaactgctgcagatgctggaaaaacagaagaaaggcactagtggcggaggctccggcggtggcattcttaaaattctgaacgaaatagcatctattggttcaactaaacagaagcaagcaattcttgaaaagaataaagataatgaattgcttaaacgagtatatcgtctgacttattctcgtgggttacagtattatatcaagaaatggcctaaacctggtattgctacccagagttttggaatgttgactcttaccgatatgcttgacttcattgaattcacattagctactcggaaattgactggaaatgcagcaattgaggaattaactggatatatcaccgatggtaaaaaagatgatgttgaagttttgcgtcgagtgatgatgcgagaccttgaatgtggtgcttcagtatctattgcaaacaaagtttggccaggtttaattcctgaacaacctcaaatgctcgcaagttcttatgatgaaaaaggcattaataagaatatcaaatttccagcctttgctcagttaaaagctgatggagctcggtgttttgctgaagttagaggtgatgaattagatgatgttcgtcttttatcacgagctggtaatgaatatctaggattagatcttcttaaggaagagttaattaaaatgaccgctgaagcccgccagattcatccagaaggtgtgttgattgatggcgaattggtataccatgagcaagttaaaaaggagccagaaggcctagattttctttttgatgcttatcctgaaaacagtaaagctaaagaattcgccgaagttgctgaatcacgtactgcttctaatggaatcgccaataaatctttaaagggaaccatttctgaaaaagaagcacaatgcatgaagtttcaggtctgggattatgtcccgttggtagaaatatacagtcttcctgcatttcgtttgaaatatgatgtacgtttttctaaactagaacaaatgacatctggatatgataaagtaattttaattgaaaaccaggtagtaaataacctagatgaagctaaggtaatttataaaaagtatattgaccaaggtcttgaaggtattattctcaaaaatatcgatggattatgggaaaatgctcgttcaaaaaatctttataaatttaaagaagtaattgatgttgatttaaaaattgtaggaatttatcctcaccgtaaagaccctactaaagcgggtggatttattcttgagtcagagtgtggaaaaattaaggtaaatgctggttcaggcttaaaagataaagccggtgtaaaatcgcatgaacttgaccgtactcgcattatggaaaaccaaaattattatattggaaaaattctagagtgcgaatgcaacggttggttaaaatctgatggccgcactgattacgttaaattatttcttccgattgcgattcgtttacgtgaagataaaactaaagctaatacattcgaagatgtatttggtgattttcatgaggtaactggtctataa

SEQ ID NO: 10 - protein, 575 aa, Artificial
  Synthetic Construct
MRGSHHHHHHTDPALRATVKFKYKGEEKEVDISKIKKVWRVGKMISFTYDEGGGKTGRGAVSEKDAPKELLQMLEKQKKGTSGGGSGGGILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 11 - DNA, 57 bp, Artificial
  Synthetic
gcactagtggcggaggctccggcggtggcgcagatggcccataccttcaaatattag

SEQ ID NO: 12 - DNA, 47 bp, Artificial
  Synthetic
atgcggccgcataggccttagctccccttctgacgtttcctctgcac

SEQ ID NO: 13 - DNA, 44 bp, Artificial
  Synthetic
gatccggccctgagggccattcttaaaattctgaacgaaatagc

SEQ ID NO: 14 - DNA, 50 bp, Artificial
  Synthetic
ccgccggagcctccgccactagtgcctagaccagttacctcatgaaaatc

SEQ ID NO: 15 - DNA, 2643 bp, Artificial
  Synthetic
ctcgagaaatcataaaaaatttatttgctttgtgagcggataacaattataatagattcaattgtgagcggataacaatttcacacagaattcattaaagaggagaaattaactatgagaggatctcaccatcaccatcaccatacggatccggccctgagggccattcttaaaattctgaacgaaatagcatctattggttcaactaaacagaagcaagcaattcttgaaaagaataaagataatgaattgcttaaacgagtatatcgtctgacttattctcgtgggttacagtattatatcaagaaatggcctaaacctggtattgctacccagagttttggaatgttgactcttaccgatatgcttgacttcattgaattcacattagctactcggaaattgactggaaatgcagcaattgaggaattaactggatatatcaccgatggtaaaaaagatgatgttgaagttttgcgtcgagtgatgatgcgagaccttgaatgtggtgcttcagtatctattgcaaacaaagtttggccaggtttaattcctgaacaacctcaaatgctcgcaagttcttatgatgaaaaaggcattaataagaatatcaaatttccagcctttgctcagttaaaagctgatggagctcggtgttttgctgaagttagaggtgatgaattagatgatgttcgtcttttatcacgagctggtaatgaatatctaggattagatcttcttaaggaagagttaattaaaatgaccgctgaagcccgccagattcatccagaaggtgtgttgattgatggcgaattggtataccatgagcaagttaaaaaggagccagaaggcctagattttctttttgatgcttatcctgaaaacagtaaagctaaagaattcgccgaagttgctgaatcacgtactgcttctaatggaatcgccaataaatctttaaagggaaccatttctgaaaaagaagcacaatgcatgaagtttcaggtctgggattatgtcccgttggtagaaatatacagtcttcctgcatttcgtttgaaatatgatgtacgtttttctaaactagaacaaatgacatctggatatgataaagtaattttaattgaaaaccaggtagtaaataacctagatgaagctaaggtaatttataaaaagtatattgaccaaggtcttgaaggtattattctcaaaaatatcgatggattatgggaaaatgctcgttcaaaaaatctttataaatttaaagaagtaattgatgttgatttaaaaattgtaggaatttatcctcaccgtaaagaccctactaaagcgggtggatttattcttgagtcagagtgtggaaaaattaaggtaaatgctggttcaggcttaaaagataaagccggtgtaaaatcgcatgaacttgaccgtactcgcattatggaaaaccaaaattattatattggaaaaattctagagtgcgaatgcaacggttggttaaaatctgatggccgcactgattacgttaaattatttcttccgattgcgattcgtttacgtgaagataaaactaaagctaatacattcgaagatgtatttggtgattttcatgaggtaactggtctaggcactagtggcggaggctccggcggtggcgcagatggcccataccttcaaatattagagcaacctaaacagagaggatttcgtttccgttatgtatgtgaaggcccatcccatggtggattacctggtgcctctagtgaaaagaacaagaagtcttaccctcaggtcaaaatctgcaactatgtgggaccagcaaaggttattgttcagttggtcacaaatggaaaaaatatccacctgcatgcccacagcctggtgggaaaacactgtgaggatgggatctgcactgtaactgctggacccaaggacatggtggtcggcttcgcaaacctgggtatacttcatgtgacaaagaaaaaagtatttgaaacactggaagcacgaatgacagaggcgtgtataaggggctataatcctggactcttggtgcaccctgaccttgcctatttgcaagcagaaggtggaggggaccggcagctgggagatcgggaaaaagagctaatccgccaagcagctctgcagcagaccaaggagatggacctcagcgtggtgcggctcatgtttacagcttttcttccggatagcactggcagcttcacaaggcgcctggaacccgtggtatcagacgccatctatgacagtaaagcccccaatgcatccaacttgaaaattgtaagaatggacaggacagctggatgtgtgactggaggggaggaaatttatcttctttgtgacaaagttcagaaagatgacatccagattcgattttatgaagaggaagaaaatggtggagtctgggaaggatttggagatttttcccccacagatgttcatagacaatttgccattgtcttcaaaactccaaagtataaagatattaatattacaaaaccagcctctgtgtttgtccagcttcggaggaaatctgacttggaaaccagtgaaccaaaacctttcctctactatcctgaaatcaaagataaagaagaagtgcagaggaaacgtcagaaggggagctaa

SEQ ID NO: 16 - protein, 842 aa, Artificial
  Synthetic Construct
MRGSHHHHHHTDPALRAILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGLGTSGGGSGGGADGPYLQILEQPKQRGFRFRYVCEGPSHGGLPGASSEKNKKSYPQVKICNYVGPAKVIVQLVTNGKNIHLHAHSLVGKHCEDGICTVTAGPKDMVVGFANLGILHVTKKKVFETLEARMTEACIRGYNPGLLVHPDLAYLQAEGGGDRQLGDREKELIRQAALQQTKEMDLSVVRLMFTAFLPDSTGSFTRRLEPVVSDAIYDSKAPNASNLKIVRMDRTAGCVTGGEEIYLLCDKVQKDDIQIRFYEEEENGGVWEGFGDFSPTDVHRQFAIVFKTPKYKDINITKPASVFVQLRRKSDLETSEPKPFLYYPEIKDKEEVQRKRQKGS

SEQ ID NO: 17 - DNA, 27 bp, Artificial
  Synthetic
ggcttcgcgacctaccgtaacactgac

SEQ ID NO: 18 - DNA, 23 bp, Artificial
  Synthetic
gccgacgccgtcgccgttttgac

SEQ ID NO: 19 - DNA, 32 bp, Artificial
  Synthetic
gataacaatttcacacagaattcattaaagag

SEQ ID NO: 20 - DNA, 27 bp, Artificial
  Synthetic
cccattaacatcaccatctaattcaac

(19) United States
(12) Patent Application Publication
(10) Pub. No.: US 2012/0214208 A1
(43) Pub. Date: Aug. 23, 2012

(54) FUSION POLYPEPTIDES AND USES THEREOF

(75) Inventors: Wayne Michael Patrick, Auckland (NZ); Robert Henry Wilson, Waitakere (NZ)

(73) Assignee: MASSEY UNIVERSITY, Palmerston North (NZ)

(21) Appl. No.: 13/496,263

(22) PCT Filed: Sep. 16, 2010
(86) PCT No.: PCT/NZ10/00187

§ 371 Date: Apr. 30, 2012

Related U.S. Application Data
(60) Provisional application No. 61/329,604, filed on Apr. 30, 2010.
(60) Provisional application No. 61/242,865, filed on Sep. 16, 2009.

(51) Int. Cl. C12P 19/34; C12N 9/96; C12N 5/10; C12N 1/21; C12N 1/15; C12N 1/19; C12N 15/62; C12N 15/63

(52) U.S. Cl. 435/91.52; 536/23.2; 435/188; 435/320.1; 435/252.3; 435/254.11; 435/254.2; 435/419; 435/348; 435/325

(57) ABSTRACT

The invention relates to fusion polypeptides comprising a polynucleotide-binding domain, such as a DNA-binding domain, and a ligase domain, such as a DNA ligase domain, methods for the production of such fusion polypeptides, and uses of the fusion polypeptides, for example in a range of molecular biological techniques as well as applications in the diagnostics, protein production, pharmaceutical, nutraceutical and medical fields.

### TECHNICAL FIELD

The present invention relates to the field of molecular biology, more particularly to fusion polypeptides and uses thereof. In particular the present invention relates to fusion polypeptides comprising a polynucleotide-binding domain, such as a DNA-binding domain, and a polynucleotide-ligase domain, such as a DNA ligase domain. Methods for the production of such fusion polypeptides, and uses of the fusion polypeptides, for example in a range of molecular biological techniques, are also provided.

### BACKGROUND OF THE INVENTION

Polynucleotide ligases, such as DNA ligases, are among the most widely used of molecular biological enzymes. A wide variety of molecular biology methodologies are reliant on the efficient activity of DNA ligase.

Ligases from a range of sources have been investigated for their application in molecular biology, and also in the growing number of industries in which molecular biological methodologies are employed, including the medical, pharmaceutical and food industries. Despite this, there has been little investigation into methods to modify the activity of ligases such as DNA ligases.

It is an object of the present invention to provide a fusion polypeptide comprising a polynucleotide ligase activity, such as a DNA ligase activity, to provide methods of using such a fusion polypeptide, or to at least provide the public with a useful choice.

### SUMMARY OF THE INVENTION

Accordingly, in a first aspect the present invention provides a method for producing a fusion polypeptide, the method comprising:
providing a host cell comprising at least one expression construct, the at least one expression construct comprising:
   at least one nucleic acid sequence encoding a polynucleotide-ligase polypeptide; and
   at least one nucleic acid sequence encoding a polynucleotide-binding polypeptide;
maintaining the host cell under conditions suitable for expression of the expression construct and for formation of a fusion polypeptide; and
separating the fusion polypeptide from the host cells.

In one embodiment the polynucleotide-ligase polypeptide is a DNA ligase polypeptide. In another embodiment the polynucleotide-ligase polypeptide is an RNA ligase polypeptide.

In one embodiment the polynucleotide-binding polypeptide is a DNA-binding polypeptide. In another embodiment the polynucleotide-binding polypeptide is an RNA-binding polypeptide. For example, in certain embodiments where the polynucleotide-ligase polypeptide is an RNA ligase polypeptide, the polynucleotide-binding polypeptide may conveniently be an RNA-binding polypeptide.

Accordingly, in one embodiment the method for producing a fusion polypeptide comprises:
providing a host cell comprising at least one expression construct, the at least one expression construct comprising:
   at least one nucleic acid sequence encoding a DNA ligase polypeptide; and
   at least one nucleic acid sequence encoding a DNA-binding polypeptide;
maintaining the host cell under conditions suitable for expression of the expression construct and for formation of a fusion polypeptide; and
separating the fusion polypeptide from the host cells.

In one embodiment the expression construct is in a high copy number vector.

In one embodiment the at least one nucleic acid sequence encoding a DNA ligase polypeptide is operably linked to a strong promoter.

In one embodiment the at least one nucleic acid sequence encoding a DNA-binding polypeptide is operably linked to a strong promoter.

In one embodiment the strong promoter is a viral promoter or a phage promoter.

In one embodiment the promoter is a phage promoter, for example a T5 phage promoter, or a T7 phage promoter.

In an alternative embodiment, the invention provides a method for producing a fusion polypeptide, the method comprising:
providing an in vitro expression system comprising at least one expression construct, the at least one expression comprising:
   at least one nucleic acid sequence encoding a polynucleotide-ligase polypeptide; and
   at least one nucleic acid sequence encoding a polynucleotide-binding polypeptide;
maintaining the expression system under conditions suitable for expression of an expression construct and for formation of a fusion polypeptide.

In certain embodiments, the method additionally comprises separating the fusion polypeptide from the expression system.

Another aspect of the present invention relates to an expression construct, the expression construct comprising:
at least one nucleic acid sequence encoding a polynucleotide-ligase polypeptide; and
at least one nucleic acid sequence encoding a polynucleotide-binding polypeptide.

In one embodiment the polynucleotide-ligase polypeptide is a DNA ligase polypeptide. In another embodiment the polynucleotide-ligase polypeptide is an RNA ligase polypeptide.

In one embodiment the polynucleotide-binding polypeptide is a DNA-binding polypeptide. In another embodiment the polynucleotide-binding polypeptide is an RNA-binding polypeptide.

Accordingly, in one embodiment the expression construct comprises:
at least one nucleic acid sequence encoding a DNA ligase polypeptide; and
at least one nucleic acid sequence encoding a DNA-binding polypeptide.

In one embodiment the expression construct encodes a fusion polypeptide comprising the DNA ligase polypeptide and the DNA-binding polypeptide.

In one embodiment the at least one nucleic acid sequence encoding the DNA ligase polypeptide and the at least one nucleic acid sequence encoding the DNA-binding polypeptide are present as a single open reading frame.

In one embodiment the at least one nucleic acid sequence encoding the DNA ligase polypeptide is operably linked to a promoter, such as a strong promoter.

In one embodiment the at least one nucleic acid sequence encoding the DNA-binding polypeptide is operably linked to a promoter, such as a strong promoter.

Another aspect of the present invention relates to a vector comprising an expression construct of the invention.

In one embodiment the vector is a high copy number vector.

In one embodiment the vector is a low copy number vector.

In one embodiment, the vector is for stable integration into a host cell genome.

Another aspect of the present invention relates to a host cell comprising an expression construct or a vector as defined above.

Another aspect of the present invention relates to a fusion polypeptide comprising at least one polynucleotide-ligase polypeptide fused to at least one polynucleotide-binding polypeptide.

In one embodiment the fusion polypeptide comprises at least one DNA ligase polypeptide fused to at least one DNA-binding polypeptide.

Another aspect of the present invention relates to a fusion polypeptide produced according to a method defined above.

Another aspect of the present invention relates to a composition comprising a fusion polypeptide, wherein the fusion polypeptide comprises at least one polynucleotide-ligase polypeptide fused to at least one polynucleotide-binding polypeptide.

In one embodiment the composition comprises a fusion polypeptide, wherein the fusion polypeptide comprises at least one DNA ligase polypeptide fused to at least one DNA-binding polypeptide.

Another aspect of the present invention relates to a composition comprising a fusion polypeptide, wherein the fusion polypeptide is produced according to a method defined above.

Another aspect of the present invention relates to a composition comprising an expression construct, vector, or host cell as defined above.

Another aspect of the present invention relates to a reagent comprising a composition as defined above.

In one embodiment, the reagent is a diagnostic reagent. In another embodiment, the reagent is a laboratory reagent.

Another aspect of the present invention relates to a kit comprising a composition as defined above.

In one embodiment, the kit is a diagnostic kit. In another embodiment, the kit is a laboratory kit. In various embodiments the kit optionally includes one or more other reagents, instructions for use, and the like.

In one embodiment, the composition comprises an homogenous population of fusion polypeptide.

In one embodiment, the composition comprises a mixed population of fusion polypeptides.

In one embodiment, the composition additionally comprises one or more of the following:
one or more polynucleotide-binding polypeptides, such as one or more DNA-binding polypeptides,
one or more polynucleotide-ligase polypeptides, such as one or more DNA ligase polypeptides,
one or more co-factors, or
one or more coenzymes.

Another aspect of the present invention relates to a method of ligating one or more nucleic acid molecules, wherein the method comprises contacting one or more nucleic acid molecules with one or more fusion polypeptides, wherein the one or more fusion polypeptides comprises at least one polynucleotide-ligase polypeptide fused to at least one polynucleotide-binding polypeptide.

In one embodiment, the method of ligating one or more nucleic acid molecules comprises contacting one or more nucleic acid molecules with one or more fusion poly-peptides, wherein the one or more fusion polypeptides comprises at least one DNA ligase polypeptide fused to at least one DNA-binding polypeptide.

In one embodiment the one or more nucleic acid molecules is a DNA molecule. In another embodiment, the one or more nucleic acid molecules are at least two DNA molecules.

In one embodiment the one or more nucleic acid molecules is one or more DNA duplexes.

In one embodiment one or more of the DNA duplexes comprises a 5′ or a 3′ overhang.

In one embodiment the one or more DNA duplexes do not comprise a 5′ or 3′ overhang.

In one embodiment, the method of ligating one or more nucleic acid molecules comprises contacting one or more nucleic acid molecules with one or more fusion polypeptides, wherein the one or more fusion polypeptides comprises at least one RNA ligase polypeptide fused to at least one RNA-binding polypeptide.

In one embodiment the one or more nucleic acid molecules is an RNA molecule. In another embodiment, the one or more nucleic acid molecules are at least two RNA molecules. In one embodiment, the one or more nucleic acid molecules are at least one DNA molecule and at least one RNA molecule.

In various embodiments, the one or more fusion polypeptides comprises at least one polynucleotide-ligase polypeptide fused to at least one RNA-binding polypeptide, or the one or more fusion polypeptides comprises at least one polynucleotide-ligase polypeptide fused to at least one DNA-binding polypeptide.

In various embodiments, the one or more fusion polypeptides comprises at least one RNA-ligase polypeptide fused to at least one polynucleotide-binding polypeptide, or the one or more fusion polypeptides comprises at least one DNA-ligase polypeptide fused to at least one polynucleotide-binding polypeptide.

Another aspect of the present invention relates to a method of catalysing the formation of a phosphodiester bond, wherein the method comprises contacting one or more nucleic acid molecules with a fusion polypeptide, wherein the fusion polypeptide comprises at least one polynucleotide-ligase polypeptide fused to at least one polynucleotide-binding polypeptide.

In one embodiment the method of catalysing the formation of a phosphodiester bond comprises contacting one or more nucleic acid molecules with a fusion polypeptide, wherein the fusion polypeptide comprises at least one DNA ligase polypeptide fused to at least one DNA-binding polypeptide.

In one embodiment the method of catalysing the formation of a phosphodiester bond comprises contacting one or more nucleic acid molecules with a fusion polypeptide, wherein the fusion polypeptide comprises at least one RNA ligase polypeptide fused to at least one RNA-binding polypeptide.

In one embodiment the phosphodiester bond is an intramolecular bond. In another embodiment, the phosphodiester bond is an intermolecular bond.

In one embodiment the method comprises ligation of one or more DNA duplexes comprising a 5′ or a 3′ overhang. Particularly contemplated are methods comprising ligation of one or more DNA duplexes with compatible overhanging termini (i.e., so called “sticky” or “cohesive-ended” ligation).

In one embodiment the method comprises ligation of one or more DNA duplexes not comprising a 5′ or a 3′ overhang (i.e., so called “blunt-ended ligation”).

In embodiments comprising ligation of one or more DNA duplexes with compatible overhanging termini, preferred fusion polypeptides may be selected from the group comprising p50-ligase, ligase-p50, NFAT-ligase, ligase-cTF, PprA-ligase, ligase-PprA, p50-LigA, and LigA-p50, with p50-ligase, ligase-cTF, ligase-PprA, p50-LigA, and LigA-p50 being particularly preferred.

In embodiments comprising ligation of one or more DNA duplexes not having a 5′ or a 3′ overhang or not having compatible termini, preferred fusion polypeptides may be selected from the group comprising p50-ligase, ligase-cTF, ligase-p50, NFAT-ligase, ligase-PprA, and LigA-p50, with p50-ligase, ligase-cTF, and ligase-PprA being particularly preferred.

Another aspect of the present invention relates to a fusion polypeptide for ligating one or more nucleic acid molecules, wherein the fusion polypeptide comprises at least one polynucleotide-ligase polypeptide fused to at least one polynucleotide-binding polypeptide.

In one embodiment the fusion polypeptide for ligating one or more nucleic acid molecules comprises at least one DNA ligase polypeptide fused to at least one DNA-binding polypeptide.

In one embodiment the fusion polypeptides are selected from the group comprising Sso7d-ligase, p50-ligase, ligase-p50, NFAT-ligase, ligase-NFAT, cTF-ligase, ligase-cTF, PprA-ligase, ligase-PprA, p50-LigA and LigA-p50, representative examples of which are described herein in the Examples.

In one embodiment the fusion polypeptide for ligating one or more nucleic acid molecules comprises at least one RNA ligase polypeptide fused to at least one RNA-binding polypeptide.

The use of a fusion polypeptide as described above in the preparation of a composition for ligating one or more nucleic acid molecules, or for catalysing the formation of a phosphodiester bond, is also specifically contemplated.

The following embodiments may relate to any of the above aspects.

In various embodiments the DNA ligase polypeptide is a prokaryotic DNA ligase, a prokaryotic DNA ligase variant, or a functional fragment thereof.

In one embodiment, the DNA ligase polypeptide is a bacterial DNA ligase, a bacterial DNA ligase variant, or a functional fragment thereof.

In one embodiment, the DNA ligase polypeptide is a viral DNA ligase, a viral DNA ligase variant, or a functional fragment thereof, including, for example, a bacteriophage DNA ligase, variant, or functional fragment thereof.

Particularly contemplated are E. coli DNA ligase polypeptides (for example, GenBank Accession No. M24278), variants or functional fragments thereof, or bacteriophage T4 DNA ligase polypeptide (for example, GenBank Accession No. X00039), variants or functional fragments thereof.

In various embodiments the DNA ligase polypeptide is a eukaryotic DNA ligase, variant, or functional fragment thereof, including a fungal DNA ligase, or a mammalian DNA ligase, or variants or functional fragments thereof. In some embodiments, the DNA ligase polypeptide is selected from the group comprising mammalian DNA ligase I, DNA ligase II, DNA ligase III including DNA ligase III in combination with DNA repair protein XRCC1, DNA ligase IV including DNA ligase IV in combination with XRCC4, or variants or functional fragments thereof.

In various embodiments the RNA ligase polypeptide is T4 RNA ligase, such as T4 RNA ligase I or T4 RNA ligase II.

In various embodiments the DNA-binding polypeptide is a sequence non-specific DNA-binding polypeptide.

In various embodiments, the DNA-binding polypeptide is selected from the group comprising chromosomal proteins, histones, HMf-like proteins, and archeal small basic DNA-binding proteins.

In particular embodiments, the DNA-binding polypeptide is selected from the group comprising
the PprA protein of Deinococcus radiodurans (GenBank Accession number BAA21374);
the mammalian NF-kappaB protein, including the NF-kappaB protein from Homo sapiens (GenBank Accession number NP_{—}003989), or one or more fragments thereof, such as the NF-kappaB p65 protein, the NF-kappaB p50 protein or a fragment comprising amino acids 40-366 of the human NF-kappaB protein;
the Ku protein from Mycobacterium tuberculosis (GenBank Accession number NP 215452);
the Sso7d protein from Sulfolobus solfataricus (GenBank Accession number NP 343889);
the Sac7d protein from Sulfolobus acidocaldarius (GenBank Accession number P13123);
the DdrA protein of Deinococcus radiodurans (as described in U.S. Pat. No. 7,550,564, incorporated herein by reference in its entirety);
the mammalian NFATc proteins, such as the NFATc1 protein from Mus musculus (GenBank accession number NP_{—}058071), or one or more functional fragments thereof, such as a fragment comprising amino acids 403-703 of the NFATc1 protein from Mus musculus, or one or more functional variants thereof;
or one or more homologues, functional variants or functional fragments thereof, or any combination of two or more thereof, such as the NFAT-Ala-p50 hybrid DNA-binding protein (referred to herein as cTF; See de Lumley et al. (2004), J. Mol. Biol. 339, 1059-1075, incorporated herein by reference in its entirety) comprising amino acids 403-579 of the NFATc from Mus musculus fused through an alanine residue to amino acids 249-366 from human NF-kappaB.

In one embodiment the DNA-binding polypeptide is a sequence-specific DNA-binding polypeptide, or a functional fragment or functional variant thereof.

In various embodiments, the DNA-binding polypeptide is a polypeptide selected from the group comprising zinc finger polypeptides, helix-turn-helix polypeptides, helix-loop-helix polypeptides, leucine zipper polypeptides, and transcription factors including Rel family transcription factors.

In various embodiments the nucleic acid sequence that codes for a fusion polypeptide comprises:
a nucleic acid sequence that codes for a DNA-binding polypeptide contiguous with the 5′ or 3′ end of the nucleic acid sequence that codes for a DNA ligase polypeptide, or
a nucleic acid sequence that codes for a DNA-binding polypeptide indirectly fused with the 5′ or 3′ end of the nucleic acid sequence that codes for a DNA ligase polypeptide, through a polynucleotide linker or spacer sequence of a desired length; or
a nucleic acid sequence that codes for a DNA-binding polypeptide that is inserted into the nucleic acid sequence that codes for a DNA ligase polypeptide, optionally through a polynucleotide linker or spacer sequence of a desired length; or
a nucleic acid sequence that codes for a DNA ligase polypeptide that is inserted into the nucleic acid sequence that codes for a DNA-binding polypeptide, optionally through a polynucleotide linker or spacer sequence of a desired length; or
a nucleic acid sequence that codes for a protease cleavage site spaced between the nucleic acid sequence that codes for a DNA-binding polypeptide and the nucleic acid sequence that codes for a DNA ligase polypeptide; or
a nucleic acid sequence that codes for a self-splicing element spaced between the nucleic acid sequence that codes for a DNA-binding polypeptide and the nucleic acid sequence that codes for a DNA ligase polypeptide; or
any combination of two or more thereof.

In various embodiments the at least one fusion polypeptide comprises:
an amino acid sequence that comprises a DNA-binding polypeptide or that comprises a DNA-binding polypeptide binding domain contiguous with the N- or C-terminal end of the amino acid sequence that comprises a DNA ligase polypeptide; or
an amino acid sequence that comprises a DNA-binding polypeptide indirectly fused with the N- or C-terminal of the amino acid sequence that comprises a DNA ligase polypeptide, through a peptide linker or spacer sequence of a desired length; or
an amino acid sequence that comprises a DNA-binding polypeptide that is inserted into the amino acid sequence that comprises a DNA ligase polypeptide, through a peptide linker or spacer sequence of a desired length; or
an amino acid sequence that comprises a protease cleavage site spaced between the amino acid sequence that comprises a DNA-binding polypeptide and the amino acid sequence that codes for a DNA ligase polypeptide; or
an amino acid sequence that comprises a self-splicing element spaced between the amino acid sequence that comprises a DNA-binding polypeptide and the amino acid sequence that codes for a DNA ligase polypeptide; or
any combination of two or more thereof.

In various embodiments the at least one fusion polypeptide has improved stability, such as improved stability at room temperature, or improved stability at 20° C.; at 19° C., at 18° C., at 17° C., at 16° C., at 15° C., at 14° C., at 13° C., at 12° C., at 11° C., at 10° C., at 9° C., at 8° C., at 7° C., at 6° C., at 5° C., at 4° C., at 3° C., at 20° C., at 2° C., at 1° C., or at 0° C. For example, the fusion polypeptide retains activity for at least about 24 hours, at least about 20 hours, about 16 hours, about 12 hours, about 11 hours, about 10, 9, 8, 7, 6, 5, 4, 3, or about 2 hours, or about 1 hour, when stored at room temperature, or at 20° C., at 19° C., at 18° C., at 17° C., at 16° C., at 15° C., at 14° C., at 13° C., at 12° C., at 11° C., at 10° C., at 9° C., at 8° C., at 7° C., at 6° C., at 5° C., at 4° C., at 3° C., at 20° C., at 2° C., at 1° C., or at 0° C.

In various embodiments the expression construct comprises a constitutive or regulatable promoter system.

In various embodiments the regulatable promoter system is an inducible or repressible promoter system.

In various embodiments the regulatable promoter system is selected from LacI, Trp, phage λ, phage RNA polymerase, and E. coli RNA polymerase promoter systems.

In one embodiment the promoter is any strong promoter known to those skilled in the art. Suitable strong promoters comprise adenoviral promoters, such as the adenoviral major late promoter; or heterologous promoters, such as the cytomegalovirus (CMV) promoter; the respiratory syncytial virus (RSV) promoter; the simian virus 40 (SV40) promoter; inducible promoters, such as the MMT promoter, the metallothionein promoter; heat shock promoters; the albumin promoter; the ApoAI promoter; human globin promoters; viral thymidine kinase promoters, such as the Herpes simplex thymidine kinase promoter; retroviral LTRs; the b-actin promoter; human growth hormone promoters; phage promoters such as the T5, T7, SP6 and T3 RNA polymerase promoters and the cauliflower mosaic 35S (CaMV 35S) promoter.

In various embodiments the promoter is a promoter having the sequence as shown in nucleotides 1-95 of SEQ ID NO 5.

In various embodiments, the fusion polypeptide comprises 10 or more contiguous amino acids from one of SEQ ID NOS 6, 8, 10, or 16. Preferably, the fusion polypeptide comprises at least 15, at least 20, more preferably at least 30, more preferably at least 40, more preferably at least 50, more preferably at least 60, more preferably at least 70, more preferably at least 80, more preferably at least 90, more preferably at least 100, more preferably at least 150, or more preferably at least 200 contiguous amino acids from one of SEQ ID NOS 6, 8, 10, or 16.

In one embodiment, the fusion polypeptide is a functional variant or functional fragment of a polypeptide comprising the sequence of one of SEQ ID NOS 6, 8, 10; or 16.

In various exemplary embodiments, the fusion polypeptide comprises at least 10 contiguous amino acids from a sequence selected from the group comprising:

amino acids 18 to 344 of SEQ ID NO. 6;

amino acids 18 to 300 of SEQ ID NO. 8;

amino acids 18 to 79 of SEQ ID NO. 10; or

amino acids 514 to 842 of SEQ ID NO. 16;

and at least 10 contiguous amino acids from a sequence selected from the group comprising:

amino acids 358 to 843 of SEQ ID NO. 6;

amino acids 311 to 796 of SEQ ID NO. 8;

amino acids 90 to 575 of SEQ ID NO. 10; or

amino acids 18 to 503 of SEQ ID NO. 16.

In various exemplary embodiments, the fusion polypeptide comprises the sequence of one of SEQ ID NOS 6, 8, 10, or 16.

In various embodiments, the invention provides an isolated, purified, or recombinant polynucleotide comprising at least 10 contiguous nucleotides from one of SEQ ID NOS 5, 7, 9, or 15.

In various exemplary embodiments, the polynucleotide comprises at least 10 contiguous nucleotides from a sequence selected from the group comprising:

nucleotides 166-1146 of SEQ ID NO. 5;

nucleotides 166-1185 of SEQ ID NO. 5;

nucleotides 166-1014 of SEQ ID NO. 7;

nucleotides 166-1044 of SEQ ID NO. 7;

nucleotides 166-351 of SEQ ID NO. 9;

nucleotides 166-381 of SEQ ID NO. 9;

nucleotides 1624-2640 of SEQ ID NO. 15; or

nucleotides 1654-2640 of SEQ ID NO. 15;

and at least 10 contiguous nucleotides from a sequence selected from the group comprising:

nucleotides 1147-2643 of SEQ ID NO. 5;

nucleotides 1186-2643 of SEQ ID NO. 5;

nucleotides 0.1015-2502 of SEQ ID NO. 7;

nucleotides 1045-2502 of SEQ ID NO. 7;

nucleotides 352-1839 of SEQ ID NO. 9;

nucleotides 382-1839 of SEQ ID NO. 9;

nucleotides 166-1623 of SEQ ID NO. 15; or

nucleotides 166-1653 of SEQ ID NO. 15.

In one embodiment, the polynucleotide comprises nucleotides 166-1146 of SEQ ID NO. 5, or the polynucleotide comprises nucleotides 166-1185 of SEQ ID NO. 5. In another embodiment, the polynucleotide comprises nucleotides 1147-2643 of SEQ ID NO. 5.

In a further embodiment, the polynucleotide comprises nucleotides 166-2643 of SEQ ID NO. 5. In an exemplary embodiment, the polynucleotide comprises the sequence of SEQ ID NO. 5.

In various embodiments, the polynucleotide comprises nucleotides 166-1014 of SEQ ID NO. 7, or the polynucleotide comprises nucleotides 166-1044 of SEQ ID NO. 7, or the polynucleotide comprises nucleotides 1015-2502 of SEQ ID NO. 7.

In an exemplary embodiment, the polynucleotide comprises nucleotides 166-2502 of SEQ ID NO. 7. In a further exemplary embodiment, the polynucleotide comprises the sequence of SEQ ID NO. 7.

In various embodiments, the polynucleotide comprises nucleotides 166-351 of SEQ ID NO. 9, or the polynucleotide comprises nucleotides 166-381 of SEQ ID NO. 9, or the polynucleotide comprises nucleotides 352-1839 of SEQ ID NO. 9.

In one exemplary embodiment, the polynucleotide comprises nucleotides 166-1839 of SEQ ID NO. 9. In a further exemplary embodiment, the polynucleotide comprises the sequence of SEQ ID NO. 9.

In various further embodiments, the polynucleotide comprises nucleotides 166-1623 of SEQ ID NO. 15, or the polynucleotide comprises nucleotides 166-1653 of SEQ ID NO. 15, or the polynucleotide comprises nucleotides 1624-2640 of SEQ ID NO. 15, or the polynucleotide comprises nucleotides 1654-2640 of SEQ ID NO. 15.

In an exemplary embodiment, the polynucleotide comprises nucleotides 166-2640 of SEQ ID NO. 15. In a further exemplary embodiment, the polynucleotide comprises the sequence of SEQ ID NO. 15.

In various embodiments the cell comprises two or more different expression constructs that each encode a different fusion polypeptide.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### DESCRIPTION OF THE DRAWINGS

Further aspects of the present invention will become apparent from the following description which is given by way of example only and with reference to the accompanying drawings.

FIG. 1a shows a representation of the gel-based in vitro ligation activity assay for cohesive-ended ligation with T4 DNA ligase fusion proteins. Samples are loaded: molecular marker (lanes 1 and 9), Sso7d-ligase (lane 2), cTF-ligase (lane 3), ligase-cTF (lane 4), p50-ligase (lane 5), ligase-p50 (lane 6), NFAT-ligase (lane 7), ligase-NFAT (lane 8), PprA-ligase (lane 10), ligase-PprA (lane 11), Ku-ligase (lane 12), ligase-ku (lane 13), T4 DNA ligase (lane 14), negative control (lane 15).

FIG. 1b shows a representation of the gel-based in vitro ligation activity assay for blunt-ended ligation with T4 DNA ligase fusion proteins. Samples are loaded the same as for FIG. 1a.

FIG. 2a shows a representation of the gel-based in vitro ligation activity assay for cohesive-ended ligation with E. coli LigA ligase fusion proteins. Samples are loaded: molecular marker (lanes 1 and 5), LigA (lane 2), LigA-p50 (lane 3), p50-LigA (lane 4), positive control (lane 6), negative control (lane 7), commercial control (lane 8).

FIG. 2b shows a representation of the gel-based in vitro ligation activity assay for blunt-ended ligation with E. coli LigA ligase fusion proteins. Samples are loaded the same as for FIG. 2a.

FIGS. 3 and 4 are graphs showing the results of quantitative PCR-based ligation activity assays as described herein in Example 5.

FIG. 5 shows a representation of the gel-based in vitro ligation activity assay for blunt-ended ligation. Samples are loaded: Sso7d-ligase (lane 1), p50-ligase (lane 2), ligase-PprA (lane 3), ligase-cTF (lane 4), T4 DNA ligase (lane 5), negative control (lane 6), positive control (lane 7), molecular marker (lane 8).

### DETAILED DESCRIPTION OF THE INVENTION

The present invention relates to fusion polypeptides and uses thereof. In particular the present invention relates to fusion polypeptides comprising a polynucleotide-ligase polypeptide, such as a DNA ligase polypeptide, fused with a polynucleotide-binding polypeptide, such as a DNA-binding polypeptide, together with methods of producing such fusions, and uses thereof in various molecular biological methods.

### 1. DEFINITIONS

The phrase “archaeal small basic DNA-binding protein” refers to a protein of usually between 50-75 amino acids having either at least about 50% identity to a natural Archaeal small basic DNA-binding protein such as Sso-7d from Sulfolobus sulfataricus or binds to antibodies generated against and specific to a native Archaeal small basic DNA-binding protein.

The term “coding region” or “open reading frame” (ORF) refers to the sense strand of a genomic DNA sequence or a cDNA sequence that is capable of producing a transcription product and/or a polypeptide under the control of appropriate regulatory sequences. The coding sequence is identified by the presence of a 5′ translation start codon and a 3′ translation stop codon. When inserted into a genetic construct, a “coding sequence” is capable of being expressed when it is operably linked to promoter and terminator sequences.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

Those skilled in the art will recognise that some polynucleotide-binding polypeptides have activity against both DNA and RNA (and indeed other polynucleotide analogues). Accordingly, the term “polynucleotide-binding polypeptide” refers to a polypeptide able to bind one or more polynucleotides, such as DNA, RNA, or analogues thereof.

The term “DNA-binding polypeptide” as used herein refers to a polypeptide able to bind to DNA, and includes polypeptides that bind to single-stranded DNA, those that bind to double-stranded DNA, and those that bind to DNA in another configuration. As described herein, the DNA-binding polypeptide may be fused to a DNA ligase polypeptide, for example the N-terminus or to the C-terminus of DNA ligase, without inactivating either the DNA-binding polypeptide or the ligase. It should be appreciated that a DNA-binding polypeptide may also bind to polynucleotides other than DNA, such as for example, RNA, or known analogues of natural nucleotides.

Those skilled in the art will recognise that some polynucleotide-ligase polypeptides have activity against both DNA and RNA (and indeed other polynucleotide analogues). Accordingly, the term “polynucleotide-ligase polypeptide” refers to a polypeptide able to catalyse the formation of a phosphodiester bond.

The term “DNA ligase polypeptide” may be used herein predominantly in respect of polypeptides exhibiting preferential activity on DNA polynucleotides, the term as used herein generally refers to a polypeptide able to catalyse the formation of a phosphodiester bond.

The term “domain” refers to a unit of a protein or protein complex, comprising a polypeptide subsequence, a complete polypeptide sequence, or a plurality of polypeptide sequences where that unit has a defined function. The function is understood to be broadly defined and can be ligand binding, catalytic activity or can have a stabilizing effect on the structure of the protein.

The term “expression construct” refers to a genetic construct that includes the necessary elements that permit transcribing the inserted polynucleotide molecule, and, optionally, translating the transcript into a polypeptide. An expression construct typically comprises in a 5′ to 3′ direction:

(1) a promoter, functional in the host cell into which the construct will be introduced,

(2) the polynucleotide to be expressed, and

(3) a terminator functional in the host cell into which the construct will be introduced.

Expression constructs of the invention may be inserted into a replicable vector for cloning or for expression, or may be incorporated into the host genome.

A “fragment” of a polypeptide is a subsequence of the polypeptide that performs a function that is required for the enzymatic or binding activity and/or provides three dimensional structure of the polypeptide.

The term “fusion polypeptide”, as used herein, refers to a polypeptide comprising two or amino acid subsequences, for example two or more polypeptide domains, fused (for example through respective amino and carboxyl residues by a peptide linkage) to form a single continuous polypeptide. It should be understood that the two or more amino acid sequences can either be directly fused or indirectly fused through their respective amino and carboxyl termini through a linker or spacer or an additional polypeptide.

In one embodiment, one of the amino acid sequences comprising the fusion polypeptide comprises a DNA ligase polypeptide. In one embodiment, one of the amino acid sequences comprising the fusion polypeptide comprises a DNA-binding polypeptide. Exemplary fusion polypeptides comprising a DNA ligase polypeptide and a DNA-binding polypeptide are presented herein in the Examples and the Sequence ID listing, and are specifically contemplated herein.

In one embodiment the amino acid subsequences of the fusion polypeptide are indirectly fused through a linker or spacer, the amino acid sequences of said fusion polypeptide arranged in the order of DNA ligase-linker-DNA-binding polypeptide or DNA-binding polypeptide-linker-DNA ligase, or DNA ligase-linker-DNA-binding polypeptide binding domain or DNA-binding polypeptide binding domain-linker-DNA ligase, for example. In other embodiments the amino acid sequences of the fusion polypeptide are indirectly fused through or comprise an additional polypeptide arranged in the order of DNA ligase-additional polypeptide-DNA-binding polypeptide or DNA ligase-additional polypeptide- DNA-binding polypeptide binding domain, or DNA ligase-linker-DNA-binding polypeptide-additional polypeptide or DNA ligase-linker-DNA-binding polypeptide binding domain-additional polypeptide. Again, both N-terminal extensions and C-terminal extensions of the polynucleotide-ligase polypeptide, such as a DNA ligase, are expressly contemplated herein.

A fusion polypeptide according to the invention may also comprise one or more polypeptide sequences inserted within the sequence of another polypeptide. For example, a polypeptide sequence such as a protease recognition sequence may be inserted into a variable region of a protein comprising a DNA-binding domain.

Conveniently, a fusion polypeptide of the invention may be encoded by a single nucleic acid sequence, wherein the nucleic acid sequence comprises at least two subsequences each encoding a polypeptide or a polypeptide domain. In certain embodiments, the at least two subsequences will be present “in frame” so as comprise a single open reading frame and thus will encode a fusion polypeptide as contemplated herein. In other embodiments, the at least two subsequences may be present “out of frame”, and may be separated by a ribosomal frame-shifting site or other sequence that promotes a shift in reading frame such that, on translation, a fusion polypeptide is formed. In certain embodiments, the at least two subsequences are contiguous. In other embodiments, such as those discussed above where the at least two polypeptides or polypeptide domains are indirectly fused through an additional polypeptide, the at least two subsequences are not contiguous.

The term “genetic construct” refers to a polynucleotide molecule, usually double-stranded DNA, which may have inserted into it another polynucleotide molecule (the insert polynucleotide molecule) such as, but not limited to, a cDNA molecule or a PCR product. A genetic construct may contain the necessary elements that permit transcribing the insert polynucleotide molecule, and, optionally, translating the transcript into a polypeptide. The insert polynucleotide molecule may be derived from the host cell, or may be derived from a different cell or organism and/or may be a recombinant polynucleotide. Once inside the host cell the genetic construct may become integrated in the host chromosomal DNA. The genetic construct may be linked to a vector.

The term “host cell” refers to a bacterial cell, a fungal cell, yeast cell, a plant cell, an insect cell or an animal cell such as a mammalian host cell that is capable of supporting expression of the expression construct.

The term “linker” or “spacer” as used herein relates to an amino acid or nucleotide sequence that indirectly fuses two or more polypeptides or two or more nucleic acid sequences encoding two or more polypeptides. In some embodiments the linker or spacer is about 1, 5, 10, 15, 20, 25, 30, 35, 40, 45, 50, 55, 60, 65, 70, 75, 80, 85, 90, 95 or about 100 amino acids or nucleotides in length. In other embodiments the linker or spacer is about 100, 125, 150, 175, 200, 225, 250, 275, 300, 325, 350, 375, 400, 450, 500, 550, 600, 650, 700, 750, 800, 850, 900, 950 or about 1000 amino acids or nucleotides in length. In still other embodiments the linker or spacer is from about 1 to about 1000 amino acids or nucleotides in length, from about 10 to about 1000, from about 50 to about 1000, from about 100 to about 1000, from about 200 to about 1000, from about 300 to about 1000, from about 400 to about 1000, from about 500 to about 1000, from about 600 to about 1000, from about 700 to about 1000, from about 800 to about 1000, or from about 900 to about 1000 amino acids or nucleotides in length.

In one embodiment the linker or spacer may comprise a restriction enzyme recognition site. In another embodiment the linker or spacer may comprise a protease cleavage recognition sequence such as enterokinase, thrombin or Factor Xa recognition sequence, or a self-splicing element such as an intein. In another embodiment the linker or spacer facilitates independent folding of the fusion polypeptides.

The term “mixed population”, as used herein, refers to two or more populations of entities, each population of entities within the mixed population differing in some respect from another population of entities within the mixed population. For example, when used in reference to a mixed population of expression constructs, this refers to two or more populations of expression constructs where each population of expression construct differs in respect of the fusion polypeptide encoded by the members of that population, or in respect of some other aspect of the construct, such as for example the identity of the promoter present in the construct. Alternatively, when used in reference to a mixed population of fusion polypeptides, this refers to two or more populations of fusion polypeptides where each population of fusion polypeptides differs in respect of the polypeptides, such as the polynucleotide-ligase polypeptide, for example the DNA ligase, or the polynucleotide-binding polypeptide, such as the DNA-binding polypeptide, the members of that population contain.

The term “nucleic acid” as used herein refers to a single- or double-stranded polymer of deoxyribonucleotide, ribonucleotide bases or known analogues of natural nucleotides, or mixtures thereof. The term includes reference to a specified sequence as well as to a sequence complementary thereto, unless otherwise indicated. The terms “nucleic acid” and “polynucleotide” are used herein interchangeably.

“Operably-linked” means that the sequence to be expressed is placed under the control of regulatory elements that include promoters, tissue-specific regulatory elements, temporal regulatory elements, enhancers, repressors and terminators.

The term “over-expression” generally refers to the production of a gene product in a host cell that exceeds levels of production in normal or non-transformed host cells. The term “overexpression” when used in relation to levels of messenger RNA preferably indicates a level of expression at least about 3-fold higher than that typically observed in a host cell in a control or non-transformed cell. More preferably the level of expression is at least about 5-fold higher, about 10-fold higher, about 15-fold higher, about 20-fold higher, about 25-fold higher, about 30-fold higher, about 35-fold higher, about 40-fold higher, about 45-fold higher, about 50-fold higher, about 55-fold higher, about 60-fold higher, about 65-fold higher, about 70-fold higher, about 75-fold higher, about 80-fold higher, about 85-fold higher, about 90-fold higher, about 95-fold higher, or about 100-fold higher or above, than typically observed in a control host cell or non-transformed cell.

Levels of mRNA are measured using any of a number of techniques known to those skilled in the art including, but not limited to, Northern blot analysis and RT-PCR, including quantitative RT-PCR.

The term “polypeptide”, as used herein, encompasses amino acid chains of any length but preferably at least 5 amino acids, including full-length proteins, in which amino acid residues are linked by covalent peptide bonds. Polypeptides of the present invention may be purified natural products, or may be produced partially or wholly using recombinant or synthetic techniques. The term may refer to a polypeptide, an aggregate of a polypeptide such as a dimer or other multimer, a fusion polypeptide, a polypeptide variant, or derivative thereof.

The term “promoter” refers to non transcribed cis-regulatory elements upstream of the coding region that regulate gene transcription. Promoters comprise cis-initiator elements which specify the transcription initiation site and conserved boxes such as the TATA box, and motifs that are bound by transcription factors.

When used in respect of a polypeptide of the invention, the phrase “retaining activity” and grammatical equivalents and derivatives thereof is intended to mean that the polypeptide still has useful ligase activity, useful polynucleotide binding activity (such as DNA-binding activity), or both useful ligase activity and useful polynucleotide-binding activity. Preferably, the retained activity is at least about 35, 40, 45, 50, 55, 60, 65, 70, 75, 80, 85, 90, 95, 99 or 100% of the original activity, and useful ranges may be selected between any of these values (for example, from about 35 to about 100%, from about 50 to about 100%, from about 60 to about 100%, from about 70 to about 100%, from about 80 to about 100%, and from about 90 to about 100%). For example, preferred polypeptides of the invention retain activity for a given storage period, for example retain at least about 25, 30, 35, 40, 45, 50, 55, 60, 65, 70, 75, 80, 85, 90, 95, 99 or 100% of the original activity of the polypeptide after about 1 hour at 4° C. Similarly, preferred compositions of the invention are capable of supporting the maintenance of useful activity of the polypeptides they comprise, and can be said to retain activity, ideally until applied using the methods contemplated herein.

As used herein, the term “improved stability” when used in relation to a polypeptide or composition of the invention means a polypeptide capable of retaining activity or a composition capable of supporting activity of the polypeptide for a given period, or under particular conditions, or both, for example 1 hour at 4° C. In certain embodiments, the retained ligase activity of a fusion polypeptide of the invention is greater than that exhibited by the native ligase polypeptide when maintained under the same conditions for the same period. In other embodiments, the retained polynucleotide-binding activity of a fusion polypeptide of the invention is greater than that exhibited by the native polynucleotide-binding polypeptide when maintained under the same conditions for the same period.

The phrase “sequence-non-specific DNA-binding domain” refers to a polypeptide domain which binds with significant affinity to DNA (and optionally other nucleic acid) in a nucleotide sequence-independent manner. For example, there is no known nucleic acid able to bind the polypeptide domain with more than 10-fold, or more than 20-fold, more than 50-fold, or more than 100-fold greater affinity than another nucleic acid with the same nucleotide composition but a different nucleotide sequence.

The phrase “sequence-specific DNA-binding domain” refers to a polypeptide domain which binds with significant affinity to DNA (and optionally other nucleic acid) in a nucleotide sequence-dependent manner. For example, there is a known nucleic acid able to bind the polypeptide domain with more than 10-fold, or more than 20-fold, more than 50-fold, or more than 100-fold greater affinity than another nucleic acid with the same nucleotide composition but a different nucleotide sequence.

The term “substance” when referred to in relation to being bound to or absorbed into or incorporated within a fusion polypeptides intended to mean a substance that is bound by a fusion partner or a substance that is able to be absorbed into or incorporated within a polymer fusion polypeptide.

The term “terminator” refers to sequences that terminate transcription, which are found in the 3′ untranslated ends of genes downstream of the translated sequence. Terminators are important determinants of mRNA stability and in some cases have been found to have spatial regulatory functions.

A “fragment” of a polynucleotide sequence provided herein is a subsequence of contiguous nucleotides that is preferably at least 15 nucleotides in length. The fragments of the invention preferably comprises at least 20 nucleotides, more preferably at least 30 nucleotides, more preferably at least 40 nucleotides, more preferably at least 50 nucleotides and most preferably at least 60 contiguous nucleotides of a polynucleotide of the invention. A fragment of a polynucleotide sequence can be used in antisense, gene silencing, triple helix or ribozyme technology, or as a primer, a probe, included in a microarray, or used in polynucleotide-based selection methods.

The term “fragment” in relation to promoter polynucleotide sequences is intended to include sequences comprising cis-elements and regions of the promoter polynucleotide sequence capable of regulating expression of a polynucleotide sequence to which the fragment is operably linked.

Preferably fragments of polynucleotide sequences of the invention comprise at least 20, more preferably at least 30, more preferably at least 40, more preferably at least 50, more preferably at least 100, more preferably at least 200, more preferably at least 300, more preferably at least 400, more preferably at least 500, more preferably at least 600, more preferably at least 700, more preferably at least 800, more preferably at least 900 and most preferably at least 1000 contiguous nucleotides of a polynucleotide of the invention.

The terms “functional variant” and “functional fragment” as used herein, for example in respect of DNA ligase(s) or DNA-binding polypeptide(s), refer to polypeptide sequences different from the specifically identified sequence(s), wherein one or more amino acid residues is deleted, substituted, or added, or a sequence comprising a fragment of the specifically identified sequence(s). Functional variants may be naturally occurring allelic variants, or non-naturally occurring variants. Functional variants may be from the same or from other species and may encompass homologues, paralogues and orthologues. Functional variants or functional fragments of the polypeptides possess one or more of the biological activities of the native specifically identified polypeptide, such as an ability to elicit one or more biological effects elicited by the native polypeptide. For example, a functional fragment of a DNA ligase will typically be able to catalyse the formation of a phosphodiester bond.

Functional variants or functional fragments may have greater or lesser activity than the native polypeptide. In one example, one or more of the biological activities of the specifically identified native polypeptide possessed by the functional variant or functional fragment may be present to a greater or lesser degree in the functional variant or functional fragment than is found in the native polypeptide. In another example, each of the biological activities of the specifically identified native polypeptide possessed by the functional variant or functional fragment is present to a greater or lesser degree in the functional variant or functional fragment than is found in the native polypeptide. In still a further example, it may be desirable to provide a functional variant or functional fragment in which one or more of the biological activities of the native polypeptide is maintained or is present to a greater degree than is found in the native polypeptide, but one or more other biological activities of the native polypeptide is not present or is present to a lesser degree than is found in the native polypeptide. Examples of such functional fragments include the NF-kappaB and NFAT DNA binding polypeptide fragments described herein.

Methods and assays to determine one or more biological effects elicited by polynucleotide-ligase polypeptides, such as DNA ligase(s), or polynucleotide-binding polypeptides, such as DNA-binding polypeptides, are well known in the art and examples are described herein, and such methods and assays can be used to identify or verify one or more functional variants or functional fragments of polynucleotide ligase(s) or polynucleotide-binding polypeptides. For example, an assay of the ability of a DNA ligase to catalyse the ligation of two linear fragments of DNA to form a single, larger fragment, such as those described herein in the Examples, is amenable to identifying one or more functional variants or functional fragments of a DNA ligase.

Examples of functional fragments include polypeptide fragments that comprise amino acid sequences that are responsible for catalytic activity, for example, sequence non-specific DNA binding, or phosphodiester bond formation.

Preferably fragments of polypeptide sequences of the invention (including those sequences specifically identified in the accompanying sequence identity listing) comprise at least 10, at least 15, at least 20, more preferably at least 30, more preferably at least 40, more preferably at least 50, more preferably at least 60, more preferably at least 70, more preferably at least 80, more preferably at least 90, more preferably at least 100, more preferably at least 150, more preferably at least 200, more preferably at least 250, more preferably at least 300, more preferably at least 350, more preferably at least 400, and most preferably at least 450 contiguous amino acids of a polypeptide of the invention.

The term “primer” refers to a short polynucleotide, usually having a free 3′OH group, that is hybridized to a template and used for priming polymerization of a polynucleotide complementary to the template. Such a primer is preferably at least 5, more preferably at least 6, more preferably at least 7, more preferably at least 8, more preferably at least 9, more preferably at least 10, more preferably at least 11, more preferably at least 12, more preferably at least 13, more preferably at least 14, more preferably at least 15, more preferably at least 16, more preferably at least 17, more preferably at least 18, more preferably at least 19, more preferably at least 20 nucleotides in length.

The term “probe” refers to a short polynucleotide that is used to detect a polynucleotide sequence that is complementary to the probe, in a hybridization-based assay. The probe may consist of a “fragment” of a polynucleotide as defined herein. Preferably such a probe is at least 5, more preferably at least 10, more preferably at least 20, more preferably at least 30, more preferably at least 40, more preferably at least 50, more preferably at least 100, more preferably at least 200, more preferably at least 300, more preferably at least 400 and most preferably at least 500 nucleotides in length.

The term “variant” as used herein refers to polynucleotide or polypeptide sequences different from the specifically identified sequences, wherein one or more nucleotides or amino acid residues is deleted, substituted, or added. Variants may be naturally occurring allelic variants, or non-naturally occurring variants. Variants may be from the same or from other species and may encompass homologues, paralogues and orthologues. In certain embodiments, variants of the polynucleotides and polypeptides possess biological activities that are the same or similar to those of the wild type polynucleotides or polypeptides. The term “variant” with reference to polynucleotides and polypeptides encompasses all forms of polynucleotides and polypeptides as defined herein.

### Polynucleotide and Polypeptide Variants

The term “polynucleotide(s),” as used herein, means a single or double-stranded deoxyribonucleotide or ribonucleotide polymer of any length but preferably at least 15 nucleotides, and include as non-limiting examples, coding and non-coding sequences of a gene, sense and antisense sequences complements, exons, introns, genomic DNA, cDNA, pre-mRNA, mRNA, rRNA, siRNA, miRNA, tRNA, ribozymes, recombinant polypeptides, isolated and purified naturally occurring DNA or RNA sequences, synthetic RNA and DNA sequences, nucleic acid probes, primers and fragments. A number of nucleic acid analogues are well known in the art and are also contemplated.

### Polynucleotide Variants

Variant polynucleotide sequences preferably exhibit at least 50%, more preferably at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a specified polynucleotide sequence. Identity is found over a comparison window of at least 20 nucleotide positions, preferably at least 50 nucleotide positions, at least 100 nucleotide positions, or over the entire length of the specified polynucleotide sequence.

Polynucleotide sequence identity can be determined in the following manner. The subject polynucleotide sequence is compared to a candidate polynucleotide sequence using BLASTN (from the BLAST suite of programs, version 2.2.10 [October 2004]) in bl2seq (Tatiana A. Tatusova, Thomas L. Madden (1999), “Blast 2 sequences—a new tool for comparing protein and nucleotide sequences”, FEMS Microbiol Lett. 174:247-250), which is publicly available from NCBI (ftp://ftp.ncbi.nih.gov/blast/). The default parameters of bl2seq are utilized except that filtering of low complexity parts should be turned off.

The identity of polynucleotide sequences may be examined using the following Unix command line parameters:

bl2seq nucleotideseq1 -j nucleotideseq2 -F F -p blastn

The parameter -F F turns off filtering of low complexity sections. The parameter -p selects the appropriate algorithm for the pair of sequences. The bl2seq program reports sequence identity as both the number and percentage of identical nucleotides in a line “Identities=”.

Polynucleotide sequence identity may also be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp. 276-277) which can be obtained from http://www.hgmp.mrc.ac.uk/Software/EMBOSS/. The European Bioinformatics Institute server also provides the facility to perform EMBOSS-needle global alignments between two sequences on line at http:/www.ebi.ac.uk/emboss/align/.

Alternatively the GAP program may be used which computes an optimal global alignment of two sequences without penalizing terminal gaps. GAP is described in the following paper: Huang, X. (1994) On Global Sequence Alignment. Computer Applications in the Biosciences 10, 227-235.

Polynucleotide variants of the present invention also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available bl2seq program from the BLAST suite of programs (version 2.2.10 [October 2004]) from NCBI (ftp://ftp.ncbi.nih.gov/blast/).

The similarity of polynucleotide sequences may be examined using the following Unix command line parameters:

bl2seq nucleotideseq1 -j nucleotideseq2 -F F -p tblastx

The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. The size of this database is set by default in the bl2seq program. For small E values, much less than one, the E value is approximately the probability of such a random match.

Variant polynucleotide sequences preferably exhibit an E value of less than 1×10⁻¹⁰, more preferably less than 1×10⁻²⁰, less than 1×10⁻³⁰, less than 1×10⁻⁴⁰, less than 1×10⁻⁵⁰, less than 1×10⁻⁶⁰, less than 1×10⁻⁷⁰, less than 1×10⁻⁸⁰, less than 1×10⁻⁹⁰, less than 1×10⁻¹⁰⁰, less than 1×10⁻¹¹⁰, less than 1×10⁻¹²⁰ or less than 1×10⁻¹²³ when compared with any one of the specifically identified sequences.

Alternatively, variant polynucleotides of the present invention hybridize to a specified polynucleotide sequence, or complements thereof under stringent conditions.

The term “hybridize under stringent conditions”, and grammatical equivalents thereof, refers to the ability of a polynucleotide molecule to hybridize to a target polynucleotide molecule (such as a target polynucleotide molecule immobilized on a DNA or RNA blot, such as a Southern blot or Northern blot) under defined conditions of temperature and salt concentration. The ability to hybridize under stringent hybridization conditions can be determined by initially hybridizing under less stringent conditions then increasing the stringency to the desired stringency.

With respect to polynucleotide molecules greater than about 100 bases in length, typical stringent hybridization conditions are no more than 25 to 30° C. (for example, 10° C.) below the melting temperature (Tm) of the native duplex (see generally, Sambrook et al., Eds, 1987, Molecular Cloning, A Laboratory Manual, 2nd Ed. Cold Spring Harbor Press; Ausubel et al., 1987, Current Protocols in Molecular Biology, Greene Publishing,). Tm for polynucleotide molecules greater than about 100 bases can be calculated by the formula Tm=81.5+0.41% (G+C)−log(Na+). (Sambrook et al., Eds, 1987, Molecular Cloning, A Laboratory Manual, 2nd Ed. Cold Spring Harbor Press; Bolton and McCarthy, 1962, PNAS 84:1390): Typical stringent conditions for polynucleotide of greater than 100 bases in length would be hybridization conditions such as prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

With respect to polynucleotide molecules having a length less than 100 bases, exemplary stringent hybridization conditions are 5 to 10° C. below Tm. On average, the Tm of a polynucleotide molecule of length less than 100 bp is reduced by approximately (500/oligonucleotide length)° C.

With respect to the DNA mimics known as peptide nucleic acids (PNAs) (Nielsen et al., Science. 1991 Dec. 6; 254(5037):149′-500) Tm values are higher than those for DNA-DNA or DNA-RNA hybrids, and can be calculated using the formula described in Giesen et al., Nucleic Acids Res. 1998 Nov. 1; 26(21):5004-6. Exemplary stringent hybridization conditions for a DNA-PNA hybrid having a length less than 100 bases are 5 to 10° C. below the Tm.

Variant polynucleotides of the present invention also encompasses polynucleotides that differ from the sequences of the invention but that, as a consequence of the degeneracy of the genetic code, encode a polypeptide having similar activity to a polypeptide encoded by a polynucleotide of the present invention. A sequence alteration that does not change the amino acid sequence of the polypeptide is a “silent variation”. Except for ATG (methionine) and TGG (tryptophan), other codons for the same amino acid may be changed by art recognized techniques, e.g., to optimize codon expression in a particular host organism.

Polynucleotide sequence alterations resulting in conservative substitutions of one or several amino acids in the encoded polypeptide sequence without significantly altering its biological activity are also included in the invention. A skilled artisan will be aware of methods for making phenotypically silent amino acid substitutions (see, e.g., Bowie et al., 1990, Science 247, 1306). In some embodiments, polynucleotide sequence alterations resulting in non-conservative amino acid substitutions desirably result in a functional variant as contemplated herein, and such sequence alterations are also included in the invention.

Variant polynucleotides due to silent variations and conservative substitutions in the encoded polypeptide sequence may be determined using the publicly available bl2seq program from the BLAST suite of programs (version 2.2.10 [October 2004]) from NCBI (ftp://ftp.ncbi.nih.gov/blast/) via the tblastx algorithm as previously described.

### Polypeptide Variants

The term “variant” with reference to polypeptides encompasses naturally occurring, recombinantly and synthetically produced polypeptides. Variant polypeptide sequences preferably exhibit at least 50%, more preferably at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to a sequence of the present invention. Identity is found over a comparison window of at least 20 amino acid positions, preferably at least 50 amino acid positions, at least 100 amino acid positions, or over the entire length of a polypeptide of the invention.

Polypeptide sequence identity can be determined in the following manner. The subject polypeptide sequence is compared to a candidate polypeptide sequence using BLASTP (from the BLAST suite of programs, version 2.2.10 [October 2004]) in bl2seq, which is publicly available from NCBI (ftp://ftp.ncbi.nih.gov/blast/). The default parameters of bl2seq are utilized except that filtering of low complexity regions should be turned off.

Polypeptide sequence identity may also be calculated over the entire length of the overlap between a candidate and subject polynucleotide sequences using global sequence alignment programs. EMBOSS-needle (available at http:/www.ebi.ac.uk/emboss/align/) and GAP (Huang, X. (1994) On Global Sequence Alignment. Computer Applications, in the Biosciences 10, 227-235) as discussed above are also suitable global sequence alignment programs for calculating polypeptide sequence identity.

Polypeptide variants of the present invention also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available bl2seq program from the BLAST suite of programs (version 2.2.10 [October 2004]) from NCBI (ftp://ftp.ncbi.nih.gov/blast/). The similarity of polypeptide sequences may be examined using the following Unix command line parameters:

bl2seq peptideseq1 -j peptideseq2-F F -p blastp

Variant polypeptide sequences preferably exhibit an E value of less than 1×10⁻¹⁰, more preferably less than 1×10⁻²⁰, less than 1×10⁻³⁰, less than 1×10⁻⁴⁰, less than 1×10⁻⁵⁰, less than 1×10⁻⁶⁰, less than 1×10⁻⁷⁰, less than 1×10⁻⁸⁰, less than 1×10⁻⁹⁰, less than 1×10⁻¹⁰⁰, less than 1×10⁻¹¹⁰, less than 1×10⁻¹²⁰ or less than 1×10⁻¹²³ when compared with any one of the specifically identified sequences.

The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match.

Conservative substitutions of one or several amino acids of a described polypeptide sequence without significantly altering its biological activity are also included in the invention. A skilled artisan will be aware of methods for making phenotypically silent amino acid substitutions (see, e.g., Bowie et al., 1990, Science 247, 1306). Likewise, functional variants resulting from substitution of one or more amino acids, including non-conservative substitutions, are included in the invention.

A polypeptide variant of the present invention also encompasses that which is produced from the nucleic acid encoding a polypeptide, but differs from the wild type polypeptide in that it is processed differently such that it has an altered amino acid sequence. For example a variant may be produced by an alternative splicing pattern of the primary RNA transcript to that which produces a wild type polypeptide.

The term “vector” refers to a polynucleotide molecule, usually double stranded DNA, which is used to transport the genetic construct into a host cell. The vector may be capable of replication in at least one additional host system, such as E. coli.

### 2. POLYNUCLEOTIDE LIGASES

Polynucleotide ligases (also referred to herein as polynucleotide-ligase polypeptides) are polypeptides that can catalyse the formation of a phosphodiester bond between the 3′ hydroxyl end of one nucleotide and the 5′ phosphate end of another nucleotide. For example, DNA ligases (also referred to herein as DNA ligase polypeptides) are polypeptides that can catalyse the formation of a phosphodiester bond between the 3′ hydroxyl end of one deoxyribose nucleotide and the 5′ phosphate end of another deoxyribose nucleotide. DNA ligases are usefully reviewed in Tomkinson et al. (2006), Chem. Rev., 106, 687-699, incorporated by reference herein in its entirety. Likewise, RNA ligases catalyse the formation of a phosphodiester bond between the 3′ hydroxyl end of one ribose nucleotide and the 5′ phosphate end of another ribose nucleotide.

### 2.1 Viral DNA Ligases

The simplest DNA ligases are those from viruses, including bacteriophages. Viral DNA ligases comprise two domains: a nucleotide-binding domain and an OB-fold domain (Tomkinson et al., 2006). Viral DNA ligases require the nucleotide cofactor adenosine-5′-triphosphate (ATP) for activity. The DNA ligase from bacteriophage T4 is commonly used for in vitro applications because it will join blunt-ended and cohesive-ended DNA termini, as well as repairing single stranded nicks in duplex DNA, RNA or DNA/RNA hybrids. Viral ligases, including the T4 DNA ligase, may be amenable for use in the present invention.

### 2.2 Prokaryotic DNA Ligases

Bacteria possess DNA ligases that require the cofactor nicotinamide adenine dinucleotide (NAD+), rather than ATP, for activity. The NAD+-dependent DNA ligases possess a core module that consists of nucleotide-binding and OB-fold domains, plus one or more additional domains that assist with DNA binding and/or catalysis (Tomkinson et al., 2006). The NAD+-dependent ligase from E. coli does not join blunt-ended DNA termini; nor does it join DNA to RNA. Therefore, it can be used for in vitro applications in which the selective ligation of cohesive ends is required. NAD+-dependent bacterial ligases, including the E. coli DNA ligase, may be amenable for use in the present invention.

### 2.3 Eukaryotic and Archaeal DNA Ligases

DNA ligases from eukaryotes and archaea are ATP-dependent, multi-domain enzymes. Eukaryote genomes each encode more than one DNA ligase. The recruitment of different ligases for different cellular roles is mediated by specific interactions with additional protein partners (Tomkinson et al., 2006). A great number of eukaryotic DNA ligases have been characterised, and may be amenable to use in the present invention. These include mammalian DNA ligases, which are generally considered to fall into the following four families: mammalian DNA ligase I, DNA ligase II (an alternatively-spliced form of DNA ligase III), DNA ligase III (including DNA ligase III in combination with DNA repair protein XRCC1), and DNA ligase IV (including DNA ligase IV in combination with XRCC4). A number of archeal DNA ligases have also been characterised, and may be amenable to use in the present invention. These include thermophilic archaeal ligases, for example the ligase from Pyrococcus furiosus, as described by Nishida et al. (2006), J. Mol. Biol. 360, 956-967.

### 2.4 RNA Ligases

RNA ligases are well known in the art, and are useful in the present inventin. The RNA ligases from bacteriophage T4 are reasonably well-characterised, and have been proposed for in vitro applications such as radioactive labeling of the 3′ termini of RNA, circularizing oligodeoxyribonucleotides and oligoribonucleotides, ligating oligomers and nicks, creating hybrid and chimeric DNA/RNA molecules, and miRNA cloning, because they exhibit reasonably broad substrate specificity. For example, T4 RNA ligase I catalyses the ATP-dependent covalent ligation of single-stranded 5′-phosphoryl termini of DNA or RNA to single-stranded 3′-hydroxyl termini of DNA or RNA. T4 RNA ligase II has similar activity to T4 RNA ligase I, but prefers double-stranded substrates. Viral ligases, including the T4 RNA ligase I and T4 RNA ligase II, together with functional fragments thereof, are amenable for use in the present invention, and.

### 3. POLYNUCLEOTIDE-BINDING POLYPEPTIDES

Polynucleotide-binding polypeptides are polypeptides that can bind to a polynucleotide, whether in a sequence-specific or in a sequence non-specific fashion. For example, DNA-binding polypeptides are polypeptides that are able to bind to DNA, including polypeptides that bind to single-stranded DNA, double-stranded DNA, or to DNA in another configuration. As those skilled in the art will appreciate, for the purposes of the present invention DNA-binding polypeptides can be broadly separated into sequence non-specific DNA-binding polypeptides, and sequence-specific DNA-binding polypeptides.

### 3.1 Sequence Non-Specific DNA-Binding Polypeptides

A sequence non-specific nucleic acid binding polypeptide, preferably a sequence non-specific DNA-binding polypeptide, is a polypeptide or defined region of a polypeptide (such as a domain) that binds to nucleic acid in a sequence-independent manner. That is, binding of the polypeptide to the nucleotide does not exhibit a significant preference for a particular nucleotide sequence.

Examples of sequence-non-specific DNA-binding polypeptides particularly suitable for use in the present invention include, but are not limited to, the PprA protein of Deinococcus radiodurans (Accession number BAA21374), the Ku protein from Mycobacterium tuberculosis (Accession number NP_{—}343889), archaeal small basic DNA binding proteins including Sac7d and Sso7d (Accession numbers P13123, and NP_{—}343889, respectively), the DdrA protein of Deinococcus radiodurans (as described in U.S. Pat. No. 7,550,564, incorporated herein by reference in its entirety); archael HMf-like proteins (Accession numbers including, but not limited to, U08838 and NP_{—}633849), and PCNA homologs (Accession numbers including, but not limited to, NP_{—}578712 and NP_{—}615084).

PprA is an approximately 32 kDa protein from Deinococcus radiodurans reported to be involved in the repair of DNA damage. In vitro, PprA preferentially binds to the ends of DNA molecules (Murakami et al. (2006), Biochimica et Biophysica Acta—Proteins and Proteomics, 1764, 20-23), and in vivo it appears to be important for recruiting DNA repair proteins to DNA break sites (Narumi et al. (2004) Molecular Microbiology, 54, 278-285).

Sso7d and Sac7d are approximately 7 kDa basic chromosomal proteins from the hyperthermophilic archaea Sulfolobus solfataricus and S. acidocaldarius, respectively. These proteins are lysine-rich and have high thermal, acid and chemical stability. They have been reported to bind DNA in a sequence-independent manner and are believed to be involved in stabilizing genomic DNA at elevated temperatures.

The HMf-like proteins are archaeal histones that reportedly share homology both in amino acid sequence and in structure with eukaryotic H4 histones. The HMf family of proteins have been reported to form stable dimers in solution, and several HMf homologs have been identified from thermothilic microorganisms.

It has been reported that a number of family B DNA polymerases interact with accessory proteins, for example to achieve efficient DNA synthesis. One class of accessory proteins is referred to as the sliding clamp. It has been suggested that multimeric clamps can form a torus-like structure able to accommodate double-stranded DNA. It has been reported that the sliding clamp interacts with the C terminus of particular DNA polymerases and helps secure these polymerases to the DNA template during synthesis.

The sliding clamp in eukarya is referred to as the proliferating cell nuclear antigen (PCNA), while similar proteins in other domains are often referred to as PCNA homologs. These homologs have marked structural similarity but limited sequence similarity. PCNA homologs have been identified from non-eukaryotic organisms, including thermophilic Archaea such as Sulfalobus solfataricus, Pyroccocus furiosus, and the like. PCNAs and PCNA homologs are useful sequence-non-specific DNA-binding polypeptides for the invention.

A sequence non-specific DNA-binding domain suitable for use in the invention binds to (preferably double-stranded) nucleic acids in a sequence-independent fashion. That is, a binding domain of the invention binds nucleic acids with significant affinity, such that any known nucleic acids of equivalent nucleotide compositions but differing sequence will bind to the domain with no more than 100-fold difference in binding. Non-specific binding can be assayed using methodology well known in the art, including, for example, filter binding assays or gel mobility shift assays, which can be performed using competitor nucleotides of the same nucleotide composition, but different nucleic acid sequence to determine specificity of binding.

Sequence non-specific nucleic acid binding polypeptides, including sequence non-specific DNA-binding polypeptides, may exhibit preference for single-stranded or for double-stranded nucleic acids. Typically, strand-specific binding polypeptides will exhibit a 10-fold or higher affinity for double-stranded or single-stranded nucleic acids, as the case may be. Those skilled in the art will recognise that for particular applications, double-stranded specific, sequence non-specific DNA-binding polypeptides may be preferred.

For example, specificity for binding to double-stranded nucleic acids can be tested using a variety of assays known to those of ordinary skill in the art. These include such assays as filter binding assays or gel-shift assays. For example, in a filter-binding assay the polypeptide to be assessed for binding activity to double-stranded DNA is pre-mixed with radio-labeled DNA, either double-stranded or single-stranded, in the appropriate buffer. The mixture is filtered through a membrane (e.g., nitrocellulose) which retains the protein and the protein-DNA complex. The amount of DNA that is retained on the filter is indicative of the quantity that bound to the protein. Binding can be quantified by a competition analysis in which binding of labeled DNA is competed by the addition of increasing amounts of unlabelled DNA. A polypeptide that binds double-stranded DNA at a 10-fold or greater affinity than single-stranded DNA is defined herein as a double-stranded DNA binding protein. Alternatively, binding activity can be assessed by a gel shift assay in which radiolabeled DNA is incubated with the test polypeptide. The protein-DNA complex will migrate slower through the gel than unbound DNA, resulting in a shifted band. The amount of binding is assessed by incubating samples with increasing amounts of double-stranded or single-stranded unlabeled DNA, and quantifying the amount of radioactivity in the shifted band.

### 3.2 Sequence Specific DNA-Binding Polypeptides

Generally, the use of DNA-binding polypeptides exhibiting a moderate to high degree of sequence specificity in the fusion polypeptides of the invention is less desirable. However, those skilled in the art will recognise that in certain embodiments, a degree of sequence specificity may be useful, for example, to improve the efficiency of ligation at sites comprising a particular sequence motif preferentially bound by the DNA-binding polypeptide. For example, high efficiency ligation vectors may be designed to be used in conjunction with a particular fusion polypeptide, wherein the ligation site includes a recognition sequence bound by the sequence-specific DNA-binding polypeptide domain of the fusion polypeptide.

A great many sequence-specific DNA-binding polypeptides are known, including, for example, transcription factors, restriction endonucleases, and polymerases. Sequence-specific DNA-binding polypeptides can be classified according to the secondary structure of their DNA-binding domain(s). Examples of characteristic DNA-binding domains include zinc finger motifs, helix-turn-helix motifs, leucine zippers, and helix-loop-helix motifs. Sequence-specific DNA-binding polypeptides comprising one or more of these domains are suitable for use in the present invention.

Examples of sequence-specific DNA-binding polypeptides particularly suitable for use in the present invention include, but are not limited to, transcription factors such as the mammalian NF-kappaB p50 protein, for example, human NF-kappaB p50 protein (Accession number NP_{—}003989), and murine NF-kappaB p50 protein (Accession number NP_{—}032715), and the mammalian NFAT proteins, for example one or more of NFATc 1, NFATc2, NFATc3, NFATc4, or NFATc5.

NF-kappaB (also known as Nuclear factor of kappa light polypeptide gene enhancer in B-cells 1) is a sequence-specific DNA-binding transcription factor from the Rel family. It has been reported that NF-kappaB p50 binds a specific consensus sequence with a dissociation constant (K_{D}) of 8 pM, and non-specific DNA about 1000 times more weakly (K_{D}=5.7 nM, de Lumley et al., 2004).

The NFAT family of transcription factors (also known as Nuclear factor of activated T-cells) consists of five members NFATc1, NFATc2, NFATc3, NFATc4, and NFAT5, and each is suitable for use as a DNA-binding polypeptide in the present invention.

In other embodiments, a functional variant of a sequence-specific DNA-binding polypeptide may be utilised. For example, functional variants which retain the high affinity binding exhibited by native sequence-specific DNA-binding polypeptides, but which no longer exhibit the same degree of sequence specificity are amenable to use in the present invention. Examples of such functional variants are known in the art, and include cTF—the NFAT-Ala-p50 hybrid DNA-binding protein described by de Lumley et al. (2004), J. Mol. Biol. 339, 1059-1075, incorporated by reference herein in its entirety. This hybrid comprises amino acids 403-579 of NFATc1 fused via an alanine residue to amino acids 249-366 of NF-kappaB. The authors report that this hybrid retains the high affinity for DNA that is characteristic of NF-kappaB, but has lost its sequence-specificity: de Lumley measured the K_{D} for the kappaB consensus sequence at 28 nM, and 40 nM for non-specific DNA binding.

### 4. EXPRESSION CONSTRUCTS

Processes for producing and using expression constructs for expression of fusion polypeptides in microorganisms, plant cells or animal cells (cellular expression systems) or in cell free expression systems, and host cells comprising expression constructs useful for forming a fusion polypeptide for use in the invention are well known in the art (e.g. Sambrook et al., 1987; Ausubel et al., 1987).

Expression constructs for use in methods of the invention may be inserted into a replicable vector for cloning or for expression, or may be incorporated into the host genome. Various vectors are publicly available. The vector may, for example, be in the form of a plasmid, cosmid, viral fusion polypeptide, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more selectable marker genes, an enhancer element, a promoter, and a transcription termination sequence. Construction of suitable vectors containing one or more of these components employs standard ligation techniques known in the art.

Both expression and cloning vectors contain a nucleic acid sequence that enables the vector to replicate in one or more selected host cells. Such sequences are well known for a variety of bacteria, yeast, and viruses.

In one embodiment the expression construct is present on a high copy number vector.

In one embodiment the high copy number vector is selected from those that may be present at 20 to 3000 copies per host cell.

In one embodiment the high copy number vector contain a high copy number origin of replication (ori), such as ColE1 or a ColE1-derived origin of replication. For example, the ColE-1 derived origin of replication may comprise the pUC19 origin of replication.

Numerous high copy number origins of replication suitable for use in the vectors of the present invention are known to those skilled in the art. These include the ColE1-derived origin of replication from pBR322 and its derivatives as well as other high copy number origins of replication, such as M13 FR ori or p15A ori. The 2μ plasmid origin is suitable for yeast, and various viral origins (SV40, polyoma, adenovirus, VSV or BPV) are useful for cloning vectors in mammalian cells.

Preferably, the high copy number origin of replication comprises the ColE1-derived pUC19 origin of replication.

Expression and cloning vectors will typically contain a selection gene, also termed a selectable marker to detect the presence of the vector in the transformed host cell. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

Selectable markers commonly used in plant transformation include the neomycin phophotransferase II gene (NPT II) which confers kanamycin resistance, the aadA gene, which confers spectinomycin and streptomycin resistance, the phosphinothricin acetyl transferase (bar gene) for Ignite (AgrEvo) and Basta (Hoechst) resistance, and the hygromycin phosphotransferase gene (hpt) for hygromycin resistance.

Examples of suitable selectable markers for mammalian cells are those that enable the identification of cells competent to take up expression constructs, such as DHFR or thymidine kinase. An appropriate host cell when wild-type DHFR is employed is the CHO cell line deficient in DHFR activity, prepared and propagated as described by Urlaub et al., 1980. A suitable selection gene for use in yeast is the trp1 gene present in the yeast plasmid YRp7 (Stinchcomb et al., 1979; Kingsman et al., 1979; Tschemper et al., 1980). The trp1 gene provides a selection marker for a mutant strain of yeast lacking the ability to grow in tryptophan, for example, ATCC No. 44076 or PEP4-1 [Jones, Genetics, 85:12 (1977)].

An expression construct useful for forming a fusion polypeptide preferably includes a promoter which controls expression of at least one nucleic acid encoding a DNA ligase, a DNA-binding polypeptide or the fusion polypeptide.

Promoters recognized by a variety of potential host cells are well known. Promoters suitable for use with prokaryotic hosts include the β-lactamase and lactose promoter systems [Chang et al., 1978; Goeddel et al., 1979), alkaline phosphatase, a tryptophan (trp) promoter system [Goeddel, Nucleic Acids Res., 8:4057 (1980); EP 36,776], and hybrid promoters such as the tac promoter [deBoer et al., 1983). Promoters for use in bacterial systems also will contain a Shine-Dalgarno (S.D.) sequence operably linked to the nucleic acid encoding a DNA ligase, a DNA ligase polypeptide or fusion polypeptide.

Examples of suitable promoting sequences for use with yeast hosts include the promoters for 3-phosphoglycerate kinase [Hitzeman et al., 1980) or other glycolytic enzymes [Hess et al., 1968; Holland, 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization.

Examples of suitable promoters for use in plant host cells, including tissue or organ of a monocot or dicot plant include cell-, tissue- and organ-specific promoters, cell cycle specific promoters, temporal promoters, inducible promoters, constitutive promoters that are active in most plant tissues, and recombinant promoters. Choice of promoter will depend upon the temporal and spatial expression of the cloned polynucleotide, so desired. The promoters may be those from the host cell, or promoters which are derived from genes of other plants, viruses, and plant pathogenic bacteria and fungi. Those skilled in the art will, without undue experimentation, be able to select promoters that are suitable for use in modifying and modulating expression constructs using genetic constructs comprising the polynucleotide sequences of the invention. Examples of constitutive plant promoters include the CaMV 35S promoter, the nopaline synthase promoter and the octopine synthase promoter, and the Ubi 1 promoter from maize. Plant promoters which are active in specific tissues, respond to internal developmental signals or external abiotic or biotic stresses are described in the scientific literature. Exemplary promoters are described, e.g., in WO 02/00894, which is herein incorporated by reference.

Examples of suitable promoters for use in insect host cells comprise those obtained from the genomes of viruses such as Baculovirus. Commercially available Baculovirus expression systems include flashBAC (Oxford Expression Technologies) and the Bac-to-Bac Baculovirus Expression System (Invitrogen).

Examples of suitable promoters for use in mammalian host cells comprise those obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of an expression construct by higher eukaryotes may be increased by inserting an enhancer sequence into the vector. Enhancers are cis-acting elements of DNA, usually about from 10 to 300 bp that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the DNA ligase, a DNA ligase polypeptide or fusion polypeptide coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the DNA ligase, a DNA ligase polypeptide or fusion polypeptide.

In one embodiment the expression construct comprises an upstream inducible promoter, such as a BAD promoter, which is induced by arabinose.

In one embodiment the expression construct comprises a constitutive or regulatable promoter system.

In one embodiment the regulatable promoter system is an inducible or repressible promoter system.

While it is frequently desirable to use strong promoters in the production of recombinant proteins, regulation of these promoters is usually essential since constitutive overproduction of heterologous proteins leads to decreases in growth rate, plasmid stability and culture viability.

A number of promoters are regulated by the interaction of a repressor protein with the operator (a region downstream from the promoter). The most well known operators are those from the lac operon and from bacteriophage lambda. An overview of regulated promoters in E. coli is provided in Table 1 of Friehs & Reardon, 1991.

A major difference between standard bacterial cultivations and those involving recombinant E. coli is the separation of the growth and production or induction phases. Recombinant protein production often takes advantage of regulated promoters to achieve high cell densities in the growth phase (when the promoter is “off” and the metabolic burden on the host cell is slight) and then high rates of heterologous protein production in the induction phase (following induction to turn the promoter “on”).

In one embodiment the regulatable promoter system is selected from LacI, Trp, phage lambda and phage RNA polymerase.

In one embodiment the promoter system is selected from the lac or Ptac promoter and the lad repressor, or the trp promoter and the TrpR repressor.

In one embodiment the LacI repressor is inactivated by addition of isopropyl-β-D-thiogalactopyranoside (IPTG) which binds to the active repressor causes dissociation from the operator, allowing expression.

In one embodiment the trp promoter system uses a synthetic media with a defined tryptophan concentration, such that when the concentration falls below a threshold level the system becomes self-inducible. In one embodiment 3-β-indole-acrylic acid may be added to inactivate the TrpR repressor.

In one embodiment the promoter system may make use of the bacteriophage lambda repressor cI. This repressor makes use of the lambda prophage and prevent expression of all the lytic genes by interacting with two operators termed OL and OR. These operators overlap with two strong promoters PL and PR respectively. In the presence of the cI repressor, binding of RNA polymerase is prevented. The cI repressor can be inactivated by UV-irradiation or treatment of the cells with mitomycin C. A more convenient way to allow expression of the recombinant polypeptide is the application of a temperature-sensitive version of the cI repressor cI857. Host cells carrying a lambda-based expression system can be grown to mid-exponential phase at low temperature and then transferred to high temperature to induce expression of the recombinant polypeptide.

A widely used expression system makes use of the phage T7 RNA polymerase which recognises only promoters found on the T7 DNA, and not promoters present on the host cell chromosome. Therefore, the expression construct may contain one of the T7 promoters (normally the promoter present in front of gene 10) to which the recombinant gene will be fused. The gene coding for the T7 RNA polymerase is either present on the expression construct, on a second compatible expression construct or integrated into the host cell chromosome. In all three cases, the gene is fused to an inducible promoter allowing its transcription and translation during the expression phase.

The E. coli strains BL21 (DE3) and BL21 (DE3) pLysS (Invitrogen, CA) are examples of host cells carrying the T7 RNA polymerase gene. Other cell strains carrying the T7 RNA polymerase gene are known in the art, such as Pseudomonas aeruginosa ADD1976 harboring the T7 RNA polymerase gene integrated into the genome (Brunschwig & Darzins, 1992).

Another promoter system suitable for use in the present invention is the T5 promoter system exemplified herein. Usefully, this promoter is recognised by the host E. coli RNA polymerase. Suitable E. coli host strains described herein in the Examples.

In one embodiment the promoter system makes use of promoters such as API or APR which may be induced or “switched on” to initiate the induction cycle by a temperature shift, such as by elevating the temperature from about 30-37° C. to 42° C. to initiate the induction cycle.

Preferred fusion polypeptides comprise at least one DNA ligase and at least one DNA-binding polypeptide.

A nucleic acid sequence encoding a fusion polypeptide for use herein comprises at least one nucleic acid encoding a polynucleotide-ligase polypeptide, such as a DNA ligase, and at least one nucleic acid encoding a polynucleotide-binding polypeptide, such as a DNA-binding polypeptide. Once expressed, the fusion polypeptide is able to form or facilitate formation of a phosphodiester bond.

In one embodiment the nucleic acid sequence encoding at least DNA ligase is indirectly fused with the nucleic acid sequence encoding a DNA-binding polypeptide through a polynucleotide linker or spacer sequence of a desired length.

In one embodiment the amino acid sequence of the fusion polypeptide comprising the at least one DNA-binding polypeptide is contiguous with the N-terminus of the amino acid sequence comprising a DNA ligase polypeptide.

In one embodiment the amino acid sequence of the fusion polypeptide comprising the at least one DNA-binding polypeptide is contiguous with the C-terminus of the amino acid sequence comprising a DNA ligase.

In one embodiment the amino acid sequence of the fusion protein comprising the at least one DNA-binding polypeptide is indirectly fused with the N-terminus of the amino acid sequence comprising a DNA ligase polypeptide through a peptide linker or spacer of a desired length, for example a linker or spacer that facilitates independent folding of the polypeptides comprising the fusion polypeptide.

In one embodiment the amino acid sequence of the fusion protein comprising the at least one DNA-binding polypeptide is indirectly fused with the C-terminus of the amino acid sequence comprising a DNA ligase polypeptide through a peptide linker or spacer of a desired length, for example a linker or spacer to facilitate independent folding of the fusion polypeptides.

One advantage of preferred fusion polypeptides according to the present invention is that the modification of the polypeptides comprising the fusion polypeptide does not affect their functionality. For example, the functionality of exemplary DNA ligases described herein is retained if a recombinant polypeptide is fused with the N-terminus or C-terminus thereof.

It should be appreciated that the arrangement of the proteins in the fusion polypeptide may be dependent on the order of gene sequences in the nucleic acid contained in the plasmid. For example, it may be desired to produce a fusion polypeptide wherein the polynucleotide-binding polypeptide, such as the DNA-binding polypeptide, is indirectly fused to the polynucleotide ligase. The term “indirectly fused” refers to a fusion polypeptide comprising a polynucleotide ligase polypeptide and a polynucleotide-binding polypeptide that are separated by an additional protein which may be any protein that is desired to be expressed in the fusion polypeptide.

In one embodiment the additional protein is selected from a DNA ligase polypeptide, a DNA-binding polypeptide, a cofactor or coenzyme, or a fusion polypeptide, or a linker or spacer to facilitate independent folding of the fusion polypeptides, as discussed above. In this embodiment it would be necessary to order the sequence of genes in the expression construct to reflect the desired arrangement of the fusion polypeptide.

In one embodiment the polynucleotide-binding polypeptide, such as the DNA-binding polypeptide may be directly fused to the polynucleotide-ligase polypeptide, such as the DNA ligase. The term “directly fused” is used herein to indicate where two or more peptides are linked via peptide bonds.

It may also be possible to form a composition wherein the composition comprises at least two distinct fusion polypeptides. For example, a first fusion polypeptide may comprise a single-stranded DNA-binding polypeptide fused to a DNA ligase, while a second fusion polypeptide may comprise a double-stranded DNA-binding polypeptide fused to a DNA ligase. Any combination of the fusion polypeptides described herein is possible, and may be produced so as to target a particular application. Indeed, one or more of the fusion polypeptides may show improved ligation activity towards DNA fragments with blunt-ended DNA termini, or to cohesive-ended DNA termini. Similarly, one or more of the fusion polypeptides may show improved ligation activity towards RNA fragments, or RNA-DNA hybrids. Such fusion polypeptides may be used isolation, or in combination, for example to target a particular application.

In one embodiment the expression construct is expressed in vivo. Preferably the expression construct is a plasmid which is expressed in a microorganism, preferably Escherichia coli.

In one embodiment the expression construct is expressed in vitro. Preferably the expression construct is expressed in vitro using a cell free expression system.

In one embodiment one or more genes can be inserted into a single expression construct, or one or more genes can be integrated into the host cell genome. In all cases expression can be controlled through promoters as described above.

In one embodiment the expression construct further encodes at least one additional polypeptide, optionally a fusion polypeptide comprising a polynucleotide-binding polypeptide, such as a DNA-binding polypeptide, and a polynucleotide-ligase polypeptide, such as a DNA ligase polypeptide, as discussed above.

In various embodiments, the expression construct includes one or more polypeptide tags to facilitate purification of the expressed polypeptide of the invention. Examples of such tags are well known in the art, and include polyhistidine tags, FLAG epitopes, c-myc epitopes, and the like. Methods of purifying polypeptides carrying such purification aids are also well known in the art, and include chromatography, for example in the case of polyhistidine tags immobilized metal affinity chromatography including that reliant on nickel or cobalt binding.

Methods of removing such purification aids from the expressed protein are also well known in the art. For example, the tag or epitope may be separated from the polypeptide of interest by an endopeptidase recognition sequence, an intein splice site, or any other amino acid sequence that facilitates removal of the polyhistidine-tag using endopeptidases. For terminally-tagged polypeptides, exopeptidases may conveniently be used—for example, exopeptidases such as TAGZyme (Qiagen) may be used to remove N-terminal polyhistidine tags from the expressed polypeptide.

### 5. HOST CELLS

The fusion polypeptides of the present invention are conveniently produced in a host cell, using one or more expression constructs as herein described. A fusion polypeptide of the invention can be produced by enabling the host cell to express the expression construct. This can be achieved by first introducing the expression construct into the host cell or a progenitor of the host cell, for example by transforming or transfecting a host cell or a progenitor of the host cell with the expression construct, or by otherwise ensuring the expression construct is present in the host cell.

Following transformation, the transformed host cell is maintained under conditions suitable for expression of the fusion polypeptides from the expression constructs and for formation of a fusion polypeptide. Such conditions comprise those suitable for expression of the chosen expression construct, such as a plasmid in a suitable organism, as are known in the art. For example, and particularly when high yield or overexpression is desired, provision of a suitable culture media allows the synthesis of the fusion polypeptide.

Accordingly, the present invention provides a method for producing a fusion polypeptide, the method comprising:
providing a host cell comprising at least one expression construct, the expression construct comprising:
   at least one nucleic acid sequence encoding a polynucleotide-ligase polypeptide, such as a DNA ligase polypeptide; and
   at least one nucleic acid sequence encoding a polynucleotide-binding polypeptide, such as a DNA-binding polypeptide;
maintaining the host cell under conditions suitable for expression of the expression construct; and
separating the fusion polypeptide from the host.

Preferably the host cell is a bacterial cell, a fungi cell, yeast cell, a plant cell, an insect cell or an animal cell, preferably an isolated or non-human host cell. Host cells useful in methods well known in the art (e.g. Sambrook et al., 1987; Ausubel et al., 1987) for the production of recombinant fusion polypeptides are frequently suitable for use in the methods of the present invention, bearing in mind the considerations discussed herein.

Suitable prokaryote host cells comprise eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635), and DH5α-E (Invitrogen). Other suitable prokaryotic host cells include other Enterobacteriaceae such as Escherichia spp., Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Actinomycetes such as Streptomyces, Rhodococcus, Corynebacterium and Mycobaterium.

In some embodiments E. coli strain W3110 may be used because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kanr; E. coli W3110 strain 37D6, which has the complete genotype tonA ptr3 phoA E15 (argF lac)169 degP ompT rbs7 ilvG kanr; E. coli W3110 strain 40B4, which is strain 37D6 with a non-kanamycin resistant degP deletion mutation.

In some embodiments, bacterial hosts that do not produce or produce low levels of lipopolysaccharide endotoxins may be preferably used. For example, Lactococcus lactis strains, including Lactococcus lactis strain MG1363 and Lactococcus lactis subspecies cremoris NZ9000, may be used.

In addition to prokaryotes, eukaryotic microbes such as filamentous fungi or yeast are suitable cloning or expression hosts for use in the methods of the invention. Saccharomyces cerevisiae is a commonly used eukaryotic host microorganism. Others include Schizosaccharomyces pombe (Beach and Nurse, 1981; EP 139,383), Kluyveromyces hosts (U.S. Pat. No. 4,943,529; Fleer et al., 1991) such as, e.g., K. lactis (MW98-8C, CBS683, CBS4574; Louvencourt et al., 1983), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al, 1990), K. thermotolerans, and K. marxianus; yarrowia (EP 402,226); Pichia pastoris (EP 183,070; Sreekrishna et al., 1988); Candida; Trichoderma reesia (EP 244,234); Neurospora crassa (Case et al., 1979); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., 1983; Tilburn et al., 1983; Yelton et al., 1984) and A. niger (Kelly and Hynes, 1985). Methylotropic yeasts are suitable herein and comprise yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in Anthony, 1982.

Examples of invertebrate host cells include insect cells such as Drosophila S2 and Spodoptera Sf9, as well as plant cells, such as cell cultures of cotton, corn, potato, soybean, petunia, tomato, and tobacco. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., 1980); mouse sertoli cells (TM4, Mather, 1980); monkey kidney cells (CV 1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., 1982); MRC 5 cells; FS4 cells; and a human hepatoma line (Hep G2).

Eukaryotic cell lines, and particularly mammalian cell lines, will be preferred when, for example, the DNA-binding polypeptide or the DNA ligase polypeptide requires one or more post-translational modifications, such as, for example, glycosylation. For example, one or more DNA-binding polypeptides may require post-translational modification to have optimal activity, and may thus be usefully expressed in an expression host capable of such post-translational modifications.

In one embodiment the host cell is a cell with an oxidising cytosol, for example the E. coli Origami strain (Novagen).

In another embodiment the host cell is a cell with a reducing cytosol, preferably E. coli.

The fusion polypeptide can also be formed in vitro. Preferably a cell free expression system is used. Many cell free translation systems are commercially available, and suitable for use in the production of a fusion polypeptide of the invention, bearing in mind the considerations discussed herein.

The fusion polypeptides can be purified from lysed cells using centrifugation, filtration or affinity chromatography, including immobilized metal affinity purification, where appropriate.

It will be appreciated that the expression characteristics of the fusion polypeptide may be influenced or controlled by controlling the conditions in which the fusion polypeptide is produced. This may include, for example, the conditions in which a host cell is maintained, for example temperature, the presence of substrate, and the like.

In some embodiments of the invention it is desirable to achieve overexpression of the expression constructs in the host cell. Mechanisms for overexpression a particular expression construct are well known in the art, and will depend on the construct itself, the host in which it is to be expressed, and other factors including the degree of overexpression desired or required. For example, overexpression can be achieved by i) use of a strong promoter system, for example the T5 promoter system or the T7 RNA polymerase promoter system in prokaryotic hosts; ii) use of a high copy number plasmid, for example a plasmid containing the colE1 origin of replication or iii) stabilisation of the messenger RNA, for example through use of fusion sequences, or iv) optimization of translation through, for example, optimization of codon usage, of ribosomal binding sites, or termination sites, and the like. The benefits of overexpression may allow the production of a higher yield of fusion polypeptide.

### 6. USES OF THE FUSION POLYPEPTIDES OF THE INVENTION

The invention provides fusion polypeptides exhibiting one or more improved activities, including an improved efficiency in binding to nucleic acid or in catalysing phosphodiester bond formation, or exhibiting one or more improved characteristics, such as improved stability, improved resistance to denaturation, degradation or inactivation, or exhibiting both improved activity and improved characteristics. As a consequence, the fusion polypeptides of the invention have utility in any application where phosphodiester bond formation is desirable or required. Exemplary, non-limiting examples of the uses to which the fusion polypeptides of the invention can be put include the following.

### Cloning

Cloning is the art-recognised term for the suite of techniques utilised by molecular biologists when replicating and/or recombining nucleic acid sequences, for example, to create an expression vector able to support the production of a recombinant protein, or to facilitate DNA sequencing, etc. Cloning is used in a wide array of applications ranging from gene identification, protein characterisation, genetic fingerprinting, through to large scale protein production. A great variety of specialised vectors, into which nucleic acid fragments of interest may be cloned, exist, that allow protein expression, tagging, single stranded RNA and DNA production and a host of other manipulations. Cloning of any DNA fragment essentially involves four steps: 1) fragmentation—the breaking apart of a strand or duplex of DNA; 2) ligation—the attaching together of the pieces of DNA; 3) transfection or transformation—inserting the newly formed pieces of DNA into host cells; 4) screening or selection—selecting out the cells that were successfully transfected with the newly formed pieces of DNA

Although these steps are invariable among cloning procedures a number of alternative routes can be selected, these are summarized as a ‘cloning strategy’.

### Ligation Bit Analysis

Ligation bit analysis has been used to determine the identity of a nucleotide at a particular polymorphic site, such as a single nucleotide polymorphism. This analysis requires two primers that hybridize to a target with a one nucleotide gap between the primers. Each of the four nucleotides is added to a separate reaction mixture containing DNA polymerase, ligase, target DNA and the primers. The polymerase adds a nucleotide to the 3′ end of the first primer that is complementary to the SNP, and the ligase then ligates the two adjacent primers together. Upon heating of the sample, if ligation has occurred, the now larger primer will remain hybridized and a signal, for example, fluorescence, can be detected. A further discussion of these methods can be found in U.S. Pat. Nos. 5,919,626; 5,945,283; 5,242,794; and 5,952,174. mRNA display

In mRNA display, a large library of mRNA variants are transcribed and translated in vitro. Each of the gene variants has a puromycin moiety covalently attached to its 3′ end. When the translating ribosome reaches the 3′ end of the mRNA template, the puromycin moiety enters the A site of the ribosome and is incorporated into the polypeptide that is being produced. The result is an mRNA-polypeptide fusion that can be used in downstream screening and selection experiments. A critical step in preparing mRNA display libraries is the ligation of the mRNA template to the 3′-puromycin oligonucleotide spacer. In this case, DNA ligase is used to ligate a single-stranded RNA molecule to a single-stranded DNA spacer, usually with the assistance of a single-stranded DNA “splint” that spans the ligation junction. A further discussion of the method can be found in Liu et al. (2000), Methods in Enzymology, 318, 268-293 and in U.S. Pat. Nos. 6,214,553 and 6,207,446.

The present invention also contemplates the preparation of kits for use in accordance with the present invention. Suitable kits include various reagents for use in accordance with the present invention in suitable containers and packaging materials, including tubes, vials, and shrink-wrapped and blow-molded packages.

Materials suitable for inclusion in an exemplary kit in accordance with the present invention comprise one or more fusion polypeptides of the invention, or one or more compositions of the invention, substrates of the fusion polypeptides of the invention, including for example one or more positive controls (examples of which are described herein), buffers, co-factors, and other reagents required for effective activity of the fusion polypeptides of the invention.

Specifically contemplated are kits comprising one or more polypeptides or compositions of the invention bound to one or more solid substrates, such as a microfluidics device, microcuvette, microarray, polymer bead, nano- or micro-particle including magnetic particles, and the like. The kit can also contain a control sample or a series of control samples which can be assayed and compared to the test sample contained. Each component of the kit can be enclosed within an individual container and all of the various containers can be within a single package, along with instructions for interpreting the results of the assays or reactions performed using the kit.

The invention consists in the foregoing and also envisages constructions of which the following gives examples only.

### EXAMPLES

### Example 1

### Construction of Plasmids and Production of Fusion Polypeptides

This example describes the construction of plasmids for the production in E. coli of fusion polypeptides comprising T4 DNA ligase (ligase) or E. coli ligase (LigA) fused to various DNA-binding polypeptides, as listed in Table 1 below. The orientation of the polypeptides comprising the ligase activity and the DNA-binding activity relative to one another is represented by the order in which the polypeptides are recited in the name of the fusion polypeptide—for example, p50-ligase refers to a fusion polypeptide comprising a p50 DNA-binding polypeptide fused to the N-terminus of a T4 DNA ligase polypeptide (optionally via a linking polypeptide), while ligase-p50 refers to a fusion polypeptide comprising a T4 DNA ligase polypeptide fused to the N-terminus of a p50 DNA-binding polypeptide (again, optionally via a linking polypeptide).

TABLE 1
Ligase-DNA binding Fusion polypeptides

| | E. coli |
|---|---|
| T4 DNA Ligase Fusion Polypeptides | DNA Ligase fusion polypeptides |
| T4 DNA Ligase (control) | LigA (control) |
| Sso7d-ligase | P50-ligA |
| P50-ligase | LigA-p50 |
| Ligase-p50 | |
| NFAT-ligase | |
| Ligase-NFAT | |
| cTF-ligase | |
| Ligase-cTF | |
| PprA-ligase | |
| Ligase-PprA | |
| Ku-ligase | |
| Ligase-ku | |

### Materials and Methods

1. Growth of Escherichia coli Strain DH5α-E

E. coli strain DH5α-E (Invitrogen) was used for all experiments. Cells were grown under standard conditions (LB medium, 37° C. incubation) except where noted below.

### 2. Construction of Plasmids

Representative plasmids and oligonucleotides used herein are listed in Table 2.

A DNA fragment encoding amino acids 40-366 of the human NF-kappaB (i.e. p50) was amplified from plasmid pRES112 in a polymerase chain reaction (PCR) with oligonucleotide primers p50_Sfi.for (SEQ ID No. 1) and p50-ligase.rev (SEQ ID No. 2). A DNA fragment encoding the T4 DNA ligase was amplified from plasmid pET14b-Ligase in a PCR with oligonucleotide primers p50-ligase.for (SEQ ID No. 3) and Ligase_Sfi.rev (SEQ ID No. 4). An overlap assembly PCR (ref: Horton et al. (1989) Gene, 77, 61-68), using primers p50_Sfi.for (SEQ ID No. 1) and Ligase_Sfi.rev (SEQ ID No. 4), was used to splice the p50 gene and the ligase gene together, resulting in a gene coding for the p50-ligase fusion polypeptide. The assembled p50-ligase gene was digested with the restriction enzyme SfiI and ligated to the expression vector pCA24N (which had been treated with the same restriction enzyme), yielding pCA24N-p50-ligase. The complete expression construct, including the T5-lac promoter and (His)₆-tag (both vector-encoded) is listed as SEQ ID No. 5, and the derived amino acid sequence of the fusion polypeptide is shown in the sequence ID listing as SEQ ID No. 6.

The pprA gene from Deinococcus radiodurans was optimized for enhanced expression in E. coli, using the Gene Designer software package (Villalobos et al. (2006), BMC Bioinformatics, 7, 285). While this did not change the amino acid sequence of the expressed protein (GenBank accession number BAA21374), it introduced 164 synonymous mutations into the sequence of the pprA gene. The optimized gene, with flanking restriction sites (BamHI and SpeI), was synthesized by DNA 2.0 (Menlo Park, Calif.) and supplied in their cloning vector, pJ204. The codon-optimized pprA gene was removed from pJ204-pprA by digestion with the restriction enzymes BamHI and SpeI. The p50 moiety was removed from pCA24N-p50-ligase by digestion with the same restriction enzymes (refer SEQ ID No. 5). Ligation of the digested pprA insert to the ligase-containing pCA24N backbone yielded pCA24N-pprA-ligase. The complete expression construct, including the T5-lac promoter and (His)₆-tag (both vector-encoded) is listed as SEQ ID No. 7, and the derived amino acid sequence of the fusion polypeptide is shown in the sequence ID listing as SEQ ID No. 8.

The sso7d gene from Sulfolobus solfataricus was optimized for enhanced expression in E. coli, using the Gene Designer software package (Villalobos et al. (2006), BMC Bioinformatics, 7, 285). While this did not change the amino acid sequence of the expressed protein (GenBank accession number NP_{—}343889), it introduced 47 synonymous mutations into the sequence of the pprA gene. Four codons were deleted from the 5′ terminus of the sso7d gene. The optimized gene, with flanking restriction sites (BamHI and SpeI), was synthesized by Integrated DNA Technologies (Coralville, Iowa) and supplied in their cloning vector, pIDTSmart. The codon-optimized sso7d gene was removed from pIDTSmart-sso7d by digestion with the restriction enzymes BamHI and SpeI. The p50 moiety was removed from pCA24N-p50-ligase by digestion with the same restriction enzymes (refer SEQ ID No. 5). Ligation of the digested sso7d insert to the ligase-containing pCA24N backbone yielded pCA24N-sso7d-ligase. The complete expression construct, including the T5-lac promoter and (His)₆-tag (both vector-encoded) is listed as SEQ ID No. 9, and the derived amino acid sequence of the fusion polypeptide is shown in the sequence ID listing as SEQ ID No. 10.

A-DNA fragment encoding amino acids 40-366 of the human NF-kappaB (i.e. p50) was amplified from plasmid pRES112 in a polymerase chain reaction (PCR) with oligonucleotide primers Ligase-p50.for (see Table 2, SEQ ID No. 11) and p50_Sfi.rev (see Table 2, SEQ ID No. 12). A DNA fragment encoding the T4 DNA ligase was amplified from plasmid pET14b-Ligase in a PCR with oligonucleotide primers Ligase_Sfi.for (see Table 2, SEQ ID No. 13) and Ligase-p50.rev (see Table 2, SEQ ID No. 14). An overlap assembly PCR (ref: Horton et al. (1989) Gene, 77, 61-68), using primers Ligase_Sfi.for (SEQ ID No. 13) and p50_Sfi.rev (SEQ ID No. 12), was used to splice the ligase gene and the p50 gene together, resulting in a gene coding for the ligase-p50 fusion polypeptide. The assembled ligase-p50 gene was digested with the restriction enzyme SfiI and ligated to the expression vector pCA24N (which had been treated with the same restriction enzyme), yielding pCA24N-ligase-p50. The complete expression construct, including the T5-lac promoter and (His)₆-tag (both vector-encoded) is listed as SEQ ID No. 15, and the derived amino acid sequence of the fusion polypeptide is shown in the sequence ID listing as SEQ ID No. 16.

TABLE 2
Plasmids and Oligonucleotides

| Plasmids | Description |
|---|---|
| pRES112 | “Plasmid display” vector (ref. Patrick and Blackburn (2005), |
| | FEBS J. 272, 3684-3697) containing the gene for amino acids |
| | 40-366 of human NF-kappaB p50. |
| pET14b-Ligase | Protein expression vector from Novagen, containing the cloned |
| | T4 DNA ligase gene. |
| pCA24N | Expression vector containing an IPTG-inducible T5 promoter |
| | and a (His)₆ tag (plus short linker) for high-level protein |
| | expression and purification (ref: Kitagawa et al. (2005), DNA |
| | Res. 12, 291-299). |
| pCA24N-p50- | pCA24N containing the gene that encodes the p50-ligase fusion |
| ligase | polypeptide. |
| pJ204-pprA | Cloning vector containing the codon-optimized pprA gene, |
| | synthesized by DNA 2.0 (Menlo Park, CA). |
| pCA24N-pprA- | pCA24N containing the gene that encodes the pprA-ligase |
| ligase | fusion polypeptide. |
| pIDTSmart- | Cloning vector containing the codon-optimized sso7d gene, |
| sso7d | synthesized by Integrated DNA Technologies (Coralville, IA). |
| pCA24N-sso7d- | pCA24N containing the gene that encodes the sso7d-ligase |
| ligase | fusion polypeptide. |
| pCA24N-ligase- | pCA24N containing the gene that encodes the ligase-p50 fusion |
| p50 | polypeptide. |
| Oligonucleotides | 5′ -> 3′ |
| p50_Sfi.for | GATCCGGCCCTGAGGGCCGCAGATGGCCCATACCTTCA |
| | AATATTAG [SEQ ID No. 1] |
| p50-ligase.rev | CCGCCGGAGCCTCCGCCACTAGTGCCCGAGCTCCCCTT |
| | CTGACGTTTCCTCTG [SEQ ID No. 2] |
| p50-figase.for | GCACTAGTGGCGGAGGCTCCGGCGGTGGCATTCTTAA |
| | AATTCTGAACGAAATAGCATC [SEQ ID No. 3] |
| Ligase_Sfi.rev | ATGCGGCCGCATAGGCCTTATAGACCAGTTACCTCATG |
| | AAAATC [SEQ ID No. 4] |
| Ligase-p50.for | GCACTAGTGGCGGAGGCTCCGGCGGTGGCGCAGATGG |
| | CCCATACCTTCAAATATTAG [SEQ ID No. 11] |
| p50_Sfixev | ATGCGGCCGCATAGGCCTTAGCTCCCCTTCTGACGTTT |
| | CCTCTGCAC [SEQ ID No. 12] |
| Ligase_Sfi.for | GATCCGGCCCTGAGGGCCATTCTTAAAATTCTGAACGA |
| | AATAGC [SEQ ID No. 13] |
| Ligase-p50.rev | CCGCCGGAGCCTCCGCCACTAGTGCCTAGACCAGTTAC |
| | CTCATGAAAATC [SEQ ID No. 14] |

### 3. Production and Isolation of the Fusion Polypeptide

Plasmids pCA24N-p50-ligase, pCA24N-pprA-ligase, pCA24N-sso7d-ligase and pCA24N-ligase-p50 were introduced into E. coli DH5α-E cells and the transformants were cultured in conditions suitable for the production of fusion polypeptides (28° C., with IPTG added to a concentration of 0.4 mM). Cells were pelleted, resuspended in Column Buffer (CB: 40 mM Tris-HCl, pH 8.0; 300 mM sodium chloride; 10 mM imidazole; 10% glycerol; and 1 mM beta-mercaptoethanol) and lysed by sonication. The clarified lysate was applied to a cobalt-based metal affinity resin (Talon, Clontech). After washing to remove non-(His)₆-tagged cellular proteins, the (His)₆-tagged fusion polypeptides were eluted with CB containing 150 mM imidazole. Elution fractions were pooled and dialyzed extensively against storage buffer (50 mM potassium phosphate buffer, pH 7.8; 200 mM sodium chloride; 10% glycerol).

### 4. Ligase Activity

The ligase activities of the fusion polypeptides were determined using three assays—an agarose gel-based assay (see Examples 2 and 3), a cellular transformation assay (see Example 4) and a quantitative PCR assay (see example 5).

### Example 2

### Analysis of Ligation Activity of T4 DNA Ligase Fusion Proteins

### Gel-Based Activity Assay

For cohesive-ended ligation, a 1,277 bp PCR product was generated by amplifying the plasmid pCA24N-ompC with the primers pCA24N.for (5′-GATAACAATTTCACACAGAATTCATTAAAGAG-3′, [SEQ ID No. 19]) and pCA24N.rev (5′-CCCATTAACATCACCATCTAATTCAAC-3′ [SEQ ID No. 20]). The PCR product was cleaved with the restriction enzyme SpeI, yielding two linear fragments of very similar size (638 bp and 639 bp). The two products of the cleavage reaction were co-purified and incubated in the presence or absence of various ligase proteins. 150 ng of substrate DNA was incubated with 20 pmol enzyme for 10 minutes at 16° C. The reaction was stopped by heating to 65° C. for a further 15 minutes. Ligase activities were determined by purifying the samples using Qiagen MinElute columns, and then running them on an agarose gel. Activity was measured as the appearance of the 1,277 bp ligated product, and the disappearance of the 638/639 bp substrate band.

For blunt-ended ligation, plasmid pCA24N-tig was cleaved with restriction enzymes SfiI and SmaI, yielding three linear fragments (5,232 bp, 717 bp and 589 bp). The 717 bp fragment was purified and used in the ligation assay by incubating 150 ng DNA with 20 pmol lygase enzyme for 20 minutes at 16° C. The reaction was stopped by heating to 65° C. for a further 15 minutes. Ligase activities were determined by purifying the samples using Qiagen MinElute columns, and then running them on an agarose gel. Activity was measured as the appearance of the 1,434 bp ligated product, and the disappearance of the 717 bp substrate band.

### Results

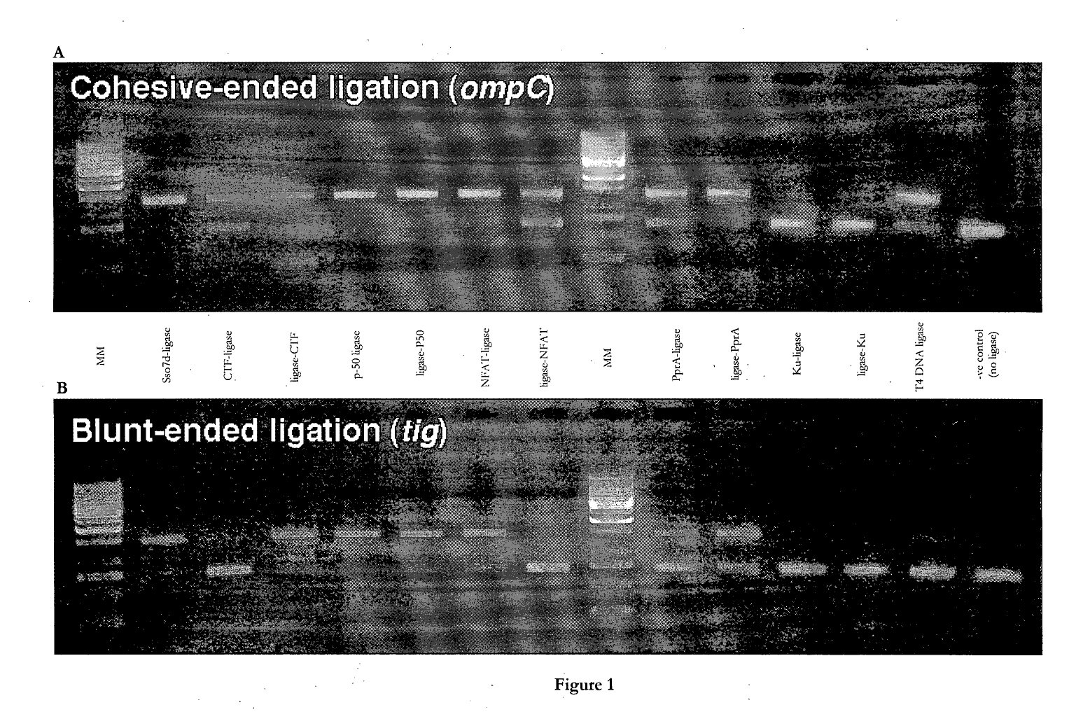

Cohesive-ended and blunt-ended ligation activity of the various fusion polypeptides is shown in FIGS. 1a and 1b, respectively. A single band (1,277 bp), as depicted in lanes 2, 4, 5, and 11 of FIG. 1a indicates highly effective cohesive-ended ligation activity with the Sso7d-ligase, ligase-cTF, p50-ligase, and ligase-PprA fusion proteins. The 1,277 bp band was also clearly evident in lanes 3, 6-8, and 10, indicating these fusion polypeptides also had robust cohesive-ended ligase activity. Ligation activity was observed with T4 DNA ligase control (FIG. 1a, lane 14), albeit less than that observed with the majority of the fusion polypeptides above.

In FIG. 1b, single bands (1,434 bp) are shown in lanes 3 and 4, indicating highly effective blunt-ended ligation activity with the ligase-cTF and p50-ligase fusion proteins. The 1,434 bp band was also clearly evident in lanes 1, 5, 6, 10 and 11, indicating these fusion polypeptides also had robust blunt-ended ligase activity. Minimal blunt-ended ligation activity was observed with T4 DNA ligase control (FIG. 1b, lane 14), markedly less than that observed with the fusion polypeptides above.

### Discussion

The results of the above gel-based assays show that the choice of fusion partner and the nature of the fusion may modulate the activity of the DNA ligase.

Specifically, for cohesive-ended ligation, fusion of T4 DNA ligase with Sso7d, cTF, p50 and PprA DNA-binding proteins exhibited markedly improved ligation activity compared to T4 DNA ligase lacking a DNA-binding protein fusion. Blunt-ended ligation activity was particularly improved when ligase was fused to cTF and p50 proteins.

### Example 3

### Analysis of Ligation Activity of E. coli LigA Fusion Proteins

### Gel-Based Activity Assay

For cohesive-ended ligation, 170 ng of the SpeI-digested ompC substrate (as described in Example 2) was incubated with 20 pmol of each LigA enzyme for 17 hours at 16° C. The reactions were heat-killed (65° C., 15 min) and run on an agarose gel. In addition to the LigA-p50 and p50-LigA fusion polypeptides, native LigA ligase and three control samples were assayed.

Positive control—commercially available T4 DNA ligase (Fermentas)

Negative control—no ligase added

Commercial control—1 μL of E. coli LigA (New England Biolabs)

For blunt-ended ligation, 120 ng of the SfiI/SmaI-digested tig substrate (as described in Example 2) was incubated with 20 pmol of each enzyme for 17 hours at 16° C. The reactions were heat-killed (65° C., 15 min), and run on an agarose gel.

### Results

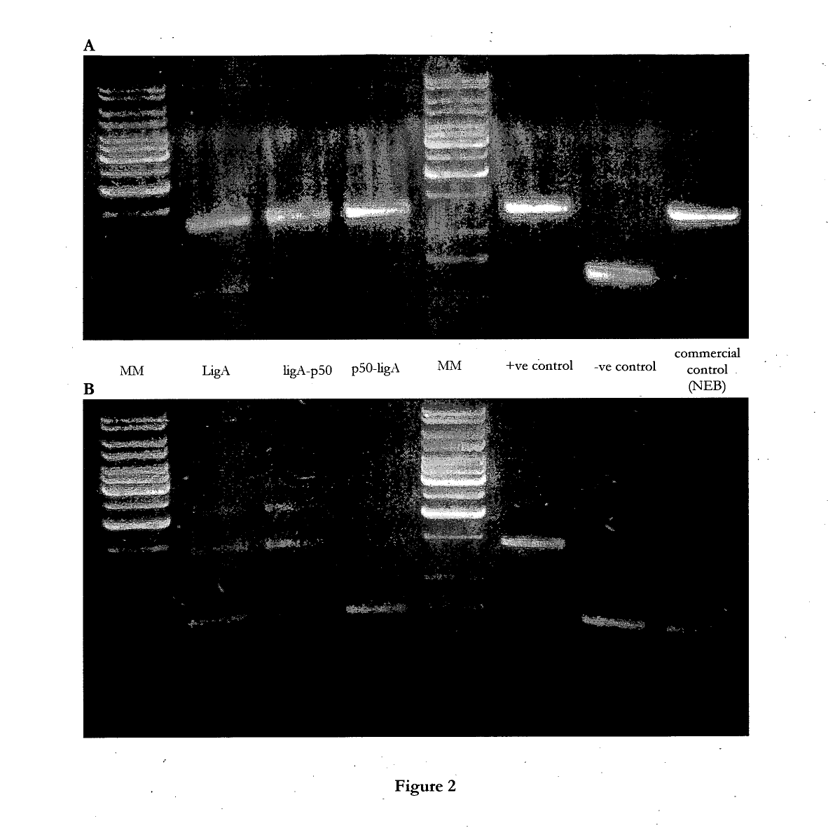

Cohesive-ended and blunt-ended ligation activity of the LigA fusion proteins is shown in FIGS. 2a and 2b, respectively. Native LigA showed comparable activity to the commercially available LigA enzyme for cohesive-ended ligation (lanes 2 and 8, FIG. 2a). Fusion to the p50 DNA-binding protein (lanes 3 and 4, FIG. 2a) showed an improvement to ligation activity, compared to unfused LigA.

As expected, the commercially available LigA enzymes showed negligible activity in the blunt-ended assay (lane 8, FIG. 2b). The native LigA showed trace activity (lane 2, FIG. 2b). Robust ligation activity in the blunt-ended assay was shown with the LigA-p50 fusion construct, but not the p50-LigA fusion.

In both cohesive-ended and blunt-ended assays, the T4 DNA ligase positive control showed good activity. No activity was observed with the negative control samples.

### Discussion

As is recognised in the art E. coli LigA exhibits reduced ligation activity when compared to T4 DNA ligase. However, fusion of a DNA-binding polypeptide to LigA improves ligation activity, and indeed the fusion of p50 DNA-binding polypeptide to the C-terminus of LigA confers on LigA blunt ended ligation activity, where no blunt-ended ligation activity is observed in the native enzyme.

### Example 4

### Transformation Assay

### Transformation Assay

The plasmid pCA24N-ompC was linearised with HindIII and SpeI restriction enzymes to produce a 5,032 bp vector backbone and a 1,311 bp insert fragment, with complementary cohesive ends. The linearized plasmid (100 ng of dephosphorylated vector and 78 ng of insert fragment) was incubated in the presence or absence of p50-ligase, ligase-PprA, Sso7d-ligase, or T4 DNA ligase, that were produced as described above. After incubation at 16° C. for 60 minutes, each sample was purified using the QiaQuick PCR Purification kit (Qiagen) and aliquots were used to transform E. coli DH5α-E cells. The transformed cells were plated on LB medium containing chloramphenicol and incubated at 37° C. overnight. The number of colonies on each plate were measured and are directly proportional to the number of recircularized plasmid molecules, and therefore to the activity of the ligase fusion protein.

### Results

The results of the transformation assay are shown in Table 3 below. The T4 DNA ligase and ligase-PprA fusion proteins were shown to out-perform the Sso7d-ligase and p50-ligase fusion proteins. An insignificant number of colonies were observed in the negative control.

TABLE 3
Transformation assay

| Ligase fusion protein | No. of colonies |
|---|---|
| T4 DNA ligase | 47 |
| Negative control (No ligase) | 4 |
| Sso7d-ligase | 18 |
| p50-ligase | 17 |
| Ligase-PprA | 53 |

### Example 5

### Analysis of Ligation Activity Using Quantitative PCR (qPCR)

This example describes the use of qPCR to quantify the ligase activities of a variety of fusion polypeptides.

### Materials and Methods

For cohesive-ended ligation, the cleaved PCR product (SpeI-digested ompC) described above in Example 2 was incubated in the presence of various ligase fusion proteins. In the first experiment, 40 ng substrate was incubated with 20 pmol of either p50-ligase, ligase-p50, PprA-ligase, Sso7d-ligase or T4 DNA ligase. In a second experiment, 420 ng of substrate was incubated with 1 pmol of either ligase-cTF, ligase-PprA, p50-ligase, or Sso7d-ligase. Following incubation at 16° C. for 10 minutes, each sample was desalted using the QiaQuick PCR Purification kit (Qiagen). A positive control reaction consisted of the PCR product and T4 DNA ligase, incubated at 16° C. for 16 hours (to allow the ligation reaction to go to completion). A negative control reaction lacked any ligase protein. The amount of ligated product in each reaction (and therefore the activity of each ligase) was measured by qPCR, using primers that amplified a 165 bp fragment which spanned the ligation site. Detection of the product in each qPCR was by binding SYBR Green (Bio-Rad). qPCR primers: ompC.for, 5′-GGCTTCGCGACCTACCGTAACACTGAC-3′ [Seq ID No 17]; ompC.rev, 5′-GCCGACGCCGTCGCCGTTTTGAC-3′ [Seq ID NO. 18].

For blunt-ended ligation, the SfiI/SmaI-digested tig substrate (as described in Example 2) was incubated with the same ligase fusion enzymes (ligase-cTF, ligase-PprA, p50-ligase, or Sso7d-ligase). For each reaction, 100 ng of substrate was incubated with 1 pmol of enzyme at 16° C. for 5 hours. The reaction was heat-killed (65° C., 15 min), the fragments purified and run on an agarose gel.

### Results

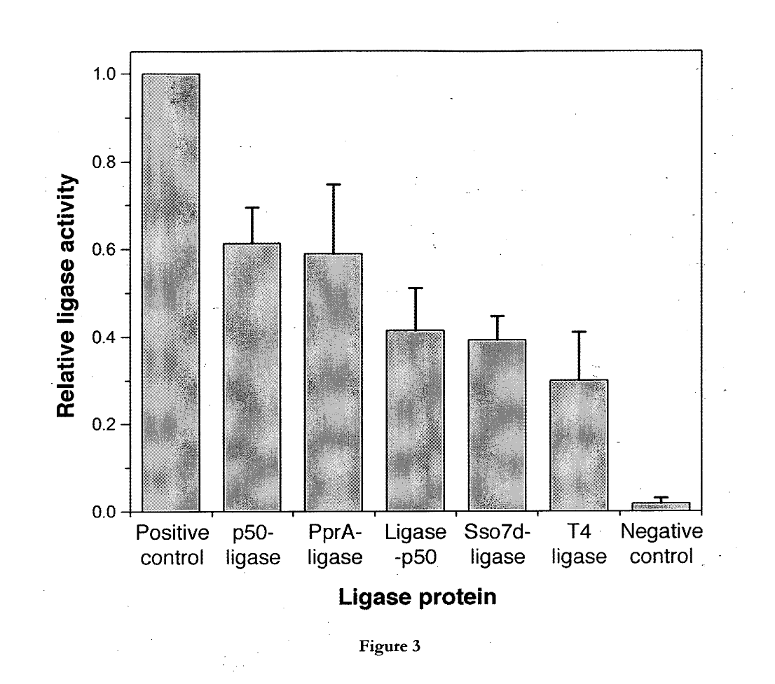

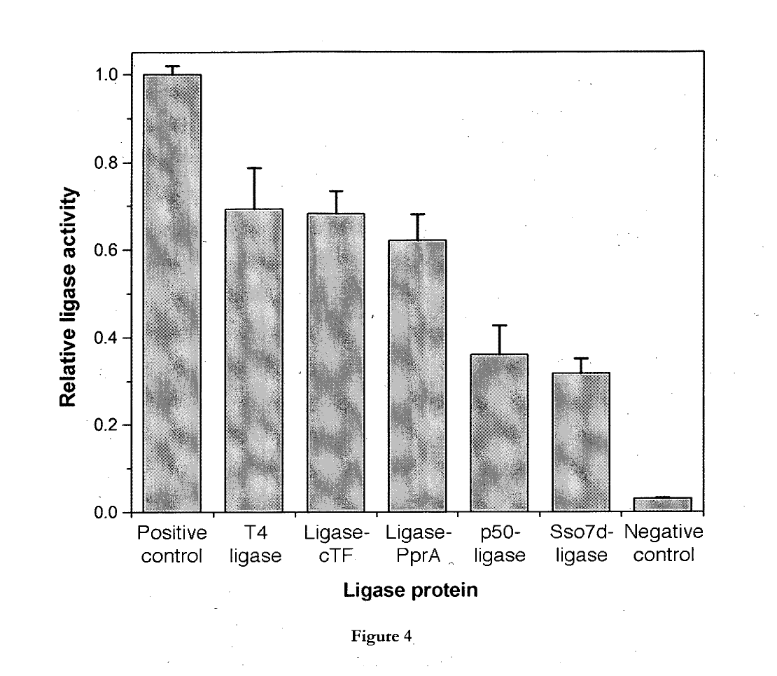

The results of the qPCR experiments are shown in FIGS. 3 and 4. The data represent the mean (+/−SEM) of three independent experiments, each of which consisted of samples assayed in triplicate. For each experiment, all activities were normalized to the activity of the positive control reaction (i.e. a ligation reaction that ran for 16 hours, rather than 10 minutes). The most active fusion proteins in experiment 1 were p50-ligase and PprA-ligase (FIG. 3), which were able to ligate approximately 60% of the substrate. In experiment 2, the most active fusion proteins were, T4 DNA ligase, ligase-cTF and ligase-PprA (FIG. 4), which were able to ligate between approximately 62% and 69% of the substrate DNA molecules In contrast, Sso7d-ligase was able to ligate approximately 30% of the substrate.

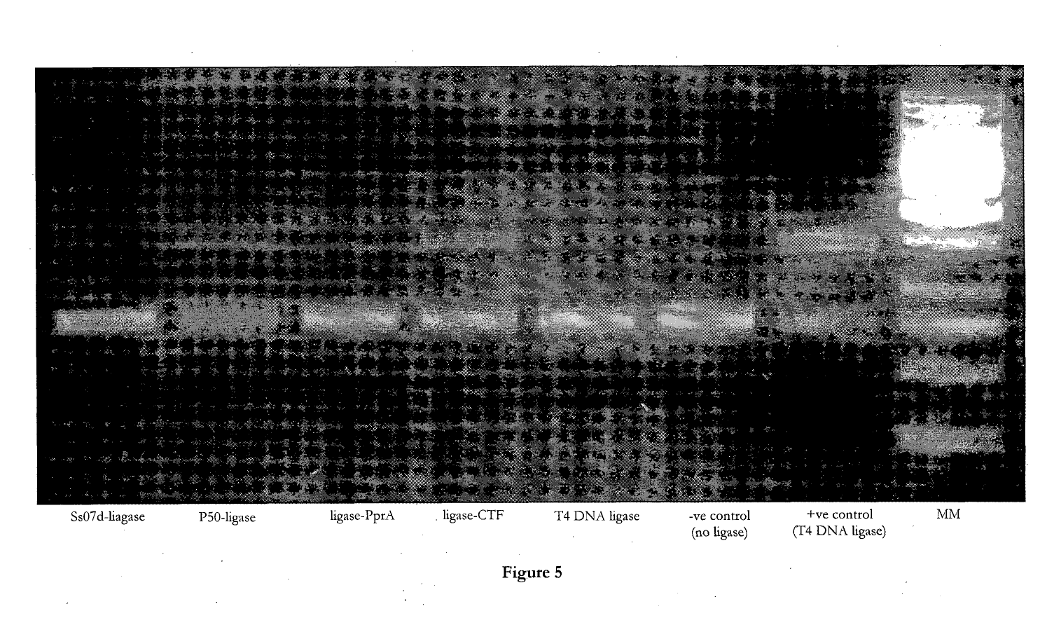

The results of the gel-based assay for blunt-ended ligation is shown in FIG. 5. Negligible ligation was observed for Sso7d-ligase (lane 1) and T4 DNA ligase (lane 5). A trace amount of ligation activity was observed for ligase-PprA (lane 3), while p50-ligase (lane 2) and ligase-cTF (lane 4) showed the greatest activity.

### Discussion

The qPCR assay described above provides further confirmation that the ligation activity of DNA ligase can be improved by its fusion to a DNA-binding polypeptide. A two-fold improvement was observed for the p50-ligase, ligase-cTF and ligase-PprA fusion polypeptides compared to ligase alone. Moreover, the nature of the fusion polypeptide—both the identity of the DNA-binding polypeptide and the orientation of the DNA-binding polypeptide relative to the ligase polypeptide—influences the ligation activity of the fusion polypeptide.

### INDUSTRIAL APPLICATION

The fusion polypeptides and methods of the present invention have utility in a wide range of molecular biological techniques, as well as application in the diagnostics, protein production, pharmaceutical, nutraceutical and medical fields.

1. An isolated, purified, or recombinant fusion polypeptide comprising at least one polynucleotide-ligase polypeptide fused to at least one polynucleotide-binding polypeptide, wherein at least one of the at least one polynucleotide-ligase polypeptide is a DNA-ligase polypeptide or an RNA-ligase polypeptide, and wherein at least one of the at least one polynucleotide-binding polypeptide is a DNA-binding polypeptide or an RNA-binding polypeptide.

2-5. (canceled)

6. The fusion polypeptide of claim 1 wherein the DNA ligase polypeptide is chosen from:
a prokaryotic DNA ligase, a prokaryotic DNA ligase variant, or a functional fragment thereof;
a viral DNA ligase, a viral DNA ligase variant, or a functional fragment thereof, including a bacteriophage DNA ligase, variant, or functional fragment thereof; and
a eukaryotic DNA ligase, functional variant, or functional fragment thereof.

7. The fusion polypeptide of claim 6 wherein:
the prokaryotic DNA ligase polypeptide is a bacterial DNA ligase, a bacterial DNA ligase variant, or a functional fragment thereof;
the viral DNA ligase polypeptide is or comprises T4 DNA ligase, or a functional variant or functional fragment thereof; or
the eukaryotic DNA ligase polypeptide is a fungal DNA ligase, a mammalian DNA ligase, of a functional variant or functional fragment thereof.

8-14. (canceled)

15. The fusion polypeptide of claim 1 wherein the DNA-binding polypeptide is chosen from chromosomal proteins, histones, HMf-like proteins, and archeal small basic DNA-binding proteins.

16. The fusion polypeptide of claim 1 wherein the DNA-binding polypeptide is chosen from:
the PprA protein of Deinococcus radiodurans (GenBank Accession number BAA21374);
the mammalian NF-kappaB protein, including the NF-kappaB protein from Homo sapiens (GenBank Accession number NP 003989), or one or more fragments thereof, such as the NF-kappaB p50 protein or a fragment comprising amino acids 40-366 of the human NF-kappaB protein;
the Ku protein from Mycobacterium tuberculosis (GenBank Accession number NP_{—}215452);
the Sso7d protein from Sulfolobus solfataricus e Bank Accession number NP_{—}343889);
the Sac7d protein from Sulfolobus acidocaldarius (GenBank Accession number P13123);
the DdrA protein of Deinococcus radiodurans; and
the mammalian NFATc proteins, such as the NFATc1 protein from Mus musculus (GenBank accession number NP 058071), or one or more functional fragments thereof including a fragment comprising amino acids 403-703 of the INFATc1 protein from Mus musculus, or one or more functional variants thereof;
or one or more homologues, functional variants or functional fragments thereof, or any combination of two or more thereof.

17. The fusion polypeptide of claim 16 wherein the DNA-binding polypeptide is the NFAT-Ala-p50 hybrid DNA-binding protein (CTF).

18. The fusion polypeptide of claim 16 wherein the DNA ligase is T4 DNA ligase and/or the DNA-binding polypeptide is chosen from PprA, Sso7d, and p50.

19. (canceled)

20. The fusion polypeptide of claim 18 comprising T4 DNA ligase and p50.

21. The fusion polypeptide of claim 1 comprising 10 or more contiguous amino acids of one of SEQ ID NOS: 6, 8, 10, or 16.

27. The fusion polypeptide of claim 21 wherein the fusion polypeptide comprises at least 10 contiguous amino acids from a sequence chosen from:
amino acids 118 to 344 of SEQ ID NO. 6;
amino acids 18 to 300 of SEQ ID NO. 8;
amino acids 18 to 79 of SEQ ID NO. 10; and
amino acids 514 to 842 of SEQ ID NO. 16;
and at least 10 contiguous amino acids from a sequence chosen from:
amino acids 358 to 843 of SEQ ID NO. 6;
amino acids 311 to 796 of SEQ ID NO. 8;
amino acids 90 to 575 of SEQ ID NO. 10; and
amino acids 18 to 503 of SEQ ID NO. 16.

23. (canceled)

24. An isolated, purified or recombinant polynucleotide encoding a fusion polypeptide as claimed in claim 1.

25. The recombinant polynucleotide of claim 24 comprising 10 or more contiguous nucleotides of one of SEQ ID NOS: 5, 7, 9, and 15.

26. The polynucleotide of claim 25, wherein the polynucleotide comprises at least 10 contiguous nucleotides from a sequence chosen from:
nucleotides 166-1 146 of SEQ ID NO. 5;
nucleotides 166-1 185 of SEQ ID NO. 5;
nucleotides 166-1014 of SEQ ID NO. 7;
nucleotides 166-1044 of SEQ ID NO. 7;
nucleotides 166-351 of SEQ ID NO. 9;
nucleotides 166-381 of SEQ ID NO. 9;
nucleotides 1624-2640 of SEQ ID NO. 15; and
nucleotides 1654-2640 of SEQ ID NO. 15;
and at least 10 contiguous nucleotides from a sequence selected the group comprising:
nucleotides 1 147-2643 of SEQ ID NO. 5;
nucleotides 1 186-2643 of SEQ ID NO. 5;
nucleotides 1015-2502 of SEQ ID NO. 7;
nucleotides 1045-2502 of SEQ ID NO. 7;
nucleotides 352-1839 of SEQ ID NO. 9;
nucleotides 382-1839 of SEQ ID NO. 9;
nucleotides 166-1623 of SEQ ID NO. 15; and
nucleotides 166-1653 of SEQ ID NO. 15.

27. An expression construct comprising the polynucleotide of claim 24.

28-33. (canceled)

34. A vector comprising an expression construct of claim 27.

35. A host cell comprising vector of claim 34.

36. A composition comprising a fusion protein as claimed in claim 1.

37. A method for producing the fusion polypeptide of claim 1, the method comprising:
growing a host cell comprising at least one expression construct, the at least one expression construct comprising:
at least one nucleic acid sequence encoding a polynucleotide-ligase polypeptide of claim 1; and
at least one nucleic acid sequence encoding a polynucleotide-binding polypeptide of claim 1;
maintaining the host cell under conditions suitable for expression of the expression construct and for formation of the fusion polypeptide; and
separating the fusion polypeptide from the host cells.

38. (canceled)

39. A method of ligating one or more nucleic acid molecules or catalysing the formation of a phosphodiester bond, comprising contacting one or more nucleic acid molecules with one or more fusion polypeptides, wherein the one or more fusion polypeptides comprises at least one polynucleotide-ligase polypeptide fused to at least one polynucleotide-binding polypeptide.

40-49. (canceled)

50. A kit comprising one or more fusion polypeptides as claimed in claim 1, optionally together with instructions for use, one or more buffers, co-factors, positive controls, negative controls, substrates, or other reagents required for activity of the fusion polypeptides.
